SEQUENCE LISTING (36 sequences)

SEQ ID NO: 1 - protein, 278 aa, Artificial Sequence
  Bacillus subtilis(Bs) flagellin
MRINHNIAALNTLNRLGSNNGAAQKNMEKLSSGLRINRAGDDAAGLAISEKMRGQIRGLEMASKNSQDGISLIQTAEGALTETHAILQRMRELTVQAGNTGTQQAEDLGAIKDEMDALIEEIDGISNRTEFNGKKLLDGTNSTDGFTFQIGANAGQQLNVKIDSMSSTALGVNALDVTDFAATAFDDQLKSIDTAINTVSTQRAKLGAVQNRLEHTINNLGASGENLTAAESRIRDVDMAKEMSEFTKNNILSQASQAMLAQANQQPQNVLQLLRPGK

SEQ ID NO: 2 - protein, 505 aa, Artificial Sequence
  Salmonella dublin(Sd) flagellin
MAQVINTNSLSLLTQNNLNKSQSSLSSAIERLSSGLRINSAKDDAAGQAIANRFTSNIKGLTQASRNANDGISIAQTTEGALNEINNNLQRVRELSVQATNGTNSDSDLKSIQDEIQQRLEEIDRVSNQTQFNGVKVLSQDNQMKIQVGANDGETITIDLQKIDVKSLGLDGFNVNGPKEATVGDLKSSFKNVTGYDTYAAGADKYRVDINSGAVVTDAVAPDKVYVNAANGQLTTDDAENNTAVDLFKTTKSTAGTAEAKAIAGAIKGGKEGDTFDYKGVTFTIDTKTGDDGNGKVSTTINGEKVTLTVADIAIGAADVNAATLQSSKNVYTSVVNGQFTFDDKTKNESAKLSDLEANNAVKGESKITVNGAEYTANATGDKITLAGKTMFIDKTASGVSTLINEDAAAAKKSTANPLASIDSALSKVDAVRSSLGAIQNRFDSAITNLGNTVTNLNSARSRIEDADYATEVSNMSKAQILQQAGTSVLAQANQVPQNVLSLLR

SEQ ID NO: 3 - protein, 387 aa, Artificial Sequence
  Pseudomonas aeruginosa (Pa) flagellin
MALTVNTNIASLNTQRNQNNSSASLNTSLQRLSTGSRINSAKDDAAGLQIANRLTSQVNGLNVATKNANDGISLAQTAEGALQQSTNILQRMRDLSLQSANGSNSDSERTALNGEVKQLQKELDRISNTTTFGGRKLLDGSFGVASFQVGSAANEIISVGIDEMSAESLNGTYFKADGGGAVTAATASGTVDIAIDITGGSAVNVKVDMKGNETAEQAAAKIAAAVNDANVGIGAFTDGAQISYVSKASADGTTSAVSGVAITDTGSTGAGTAAGTTTFTEANDTVAKIDISTAKGAQSAVLVIDEAIKQIDAQRADLGAVQNRFDNTINNLKNIGENVSAARGRIEDTDFAAETANLTKNQVLQQAGTAILAQANQLPQSVLSLLR

SEQ ID NO: 4 - protein, 550 aa, Artificial Sequence
  Shigella flexneri (Sf) flagellin
MAQVINTNSLSLITQNNINKNQSALSSSIERLSSGLRINSAKDDAAGQAIANRFTSNIKGLTQAARNANDGISVAQTTEGALSEINNNLQRIRELTVQASTGTNSDSDLDSIQDEIKSRLDEIDRVSGQTQFNGVNVLAKDGSMKIQVGANDGQTITIDLKKIDSDTLGLNGFNVNGGGAVANTAASKADLVAANATVVGNKYTVSAGYDAAKASDLLAGVSDGDTVQATINNGFGTAASATNYKYDSASKSYSFDTTTASAADVQKYLTPGVGDTAKGTITIDGSAQDVQISSDGKITASNGDKLYIDTTGRLTKNGSGASLTEASLSTLAANNTKATTIDIGGTSISFTGNSTTPDTITYSVTGAKVDQAAFDKAVSTSGNNVDFTTAGYSVNGTTGAVTKGVDSVYVDNNEALTTSDTVDFYLQDDGSVTNGSGKAVYKDADGKLTTDAETKAATTADPLKALDEAISSIDKFRSSLGAVQNRLDSAVTNLNNTTTNLSEAQSRIQDADYATEVSNMSKAQIIQQAGNSVLAKANQVPQQVLSLLQG

SEQ ID NO: 5 - protein, 498 aa, Artificial Sequence
  Escherichia coli (Ec) flagellin
MAQVINTNSLSLITQNNINKNQSALSSSIERLSSGLRINSAKDDAAGQAIANRFTSNIKGLTQAARNANDGISVAQTTEGALSEINNNLQRVRELTVQATTGTNSESDLSSIQDEIKSRLDEIDRVSGQTQFNGVNVLAKNGSMKIQVGANDNQTITIDLKQIDAKTLGLDGFSVKNNDTVTTSAPVTAFGATTTNNIKLTGITLSTEAATDTGGTNPASIEGVYTDNGNDYYAKITGGDNDGKYYAVTVANDGTVTMATGATANATVTDANTTKATTITSGGTPVQIDNTAGSATANLGAVSLVKLQDSKGNDTDTYALKDTNGNLYAADVNETTGAVSVKTITYTDSSGAASSPTAVKLGGDDGKTEVVDIDGKTYDSADLNGGNLQTGLTAGGEALTAVANGKTTDPLKALDDAIASVDKFRSSLGAVQNRLDSAVTNLNNTTTNLSEAQSRIQDADYATEVSNMSKAQIIQQAGNSVLAKANQVPQQVLSLLQG

SEQ ID NO: 6 - protein, 229 aa, Artificial Sequence
  Human IgG4 Fc region
AMVRSPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 7 - protein, 227 aa, Artificial Sequence
  Mouse IgG1 Fc region
TMVRSGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 8 - protein, 5 aa, Artificial Sequence
  Linker 1
GGGGS

SEQ ID NO: 9 - protein, 15 aa, Artificial Sequence
  Linker 2
GGGGSGGGGSGGGGS

SEQ ID NO: 10 - protein, 509 aa, Artificial Sequence
  hIgG4-Fc-Bsflagellin fusion protein
MRINHNIAALNTLNRLGSNNGAAQKNMEKLSSGLRINRAGDDAAGLAISEKMRGQIRGLEMASKNSQDGISLIQTAEGALTETHAILQRMRELTVQAGNTGTQQAEDLGAIKDEMDALIEEIDGISNRTEFNGKKLLDGTNSTDGFTFQIGANAGQQLNVKIDSMSSTALGVNALDVTDFAATAFDDQLKSIDTAINTVSTQRAKLGAVQNRLEHTINNLGASGENLTAAESRIRDVDMAKEMSEFTKNNILSQASQAMLAQANQQPQNVLQLLRGGGGSAMVRSPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 507 aa, Artificial Sequence
  mIgG1-Fc-Bsflagellin fusion protein (with linker 1)
MRINHNIAALNTLNRLGSNNGAAQKNMEKLSSGLRINRAGDDAAGLAISEKMRGQIRGLEMASKNSQDGISLIQTAEGALTETHAILQRMRELTVQAGNTGTQQAEDLGAIKDEMDALIEEIDGISNRTEFNGKKLLDGTNSTDGFTFQIGANAGQQLNVKIDSMSSTALGVNALDVTDFAATAFDDQLKSIDTAINTVSTQRAKLGAVQNRLEHTINNLGASGENLTAAESRIRDVDMAKEMSEFTKNNILSQASQAMLAQANQQPQNVLQLLRGGGGSTMVRSGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 12 - protein, 517 aa, Artificial Sequence
  mIgG1-Fc-Bsflagellin fusion protein (with linker 2)
MRINHNIAALNTLNRLGSNNGAAQKNMEKLSSGLRINRAGDDAAGLAISEKMRGQIRGLEMASKNSQDGISLIQTAEGALTETHAILQRMRELTVQAGNTGTQQAEDLGAIKDEMDALIEEIDGISNRTEFNGKKLLDGTNSTDGFTFQIGANAGQQLNVKIDSMSSTALGVNALDVTDFAATAFDDQLKSIDTAINTVSTQRAKLGAVQNRLEHTINNLGASGENLTAAESRIRDVDMAKEMSEFTKNNILSQASQAMLAQANQQPQNVLQLLRGGGGSGGGGSGGGGSTMVRSGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 13 - protein, 761 aa, Artificial Sequence
  hIgG4-Fc-Sdflagellin  fusion protein (with linker 1)
MYRMQLLSCIALSLALVTNSISMAQVINTNSLSLLTQNNLNKSQSSLSSAIERLSSGLRINSAKDDAAGQAIANRFTSNIKGLTQASRNANDGISIAQTTEGALNEINNNLQRVRELSVQATNGTNSDSDLKSIQDEIQQRLEEIDRVSNQTQFNGVKVLSQDNQMKIQVGANDGETITIDLQKIDVKSLGLDGFNVNGPKEATVGDLKSSFKNVTGYDTYAAGADKYRVDINSGAVVTDAVAPDKVYVNAANGQLTTDDAENNTAVDLFKTTKSTAGTAEAKAIAGAIKGGKEGDTFDYKGVTFTIDTKTGDDGNGKVSTTINGEKVTLTVADIAIGAADVNAATLQSSKNVYTSVVNGQFTFDDKTKNESAKLSDLEANNAVKGESKITVNGAEYTANATGDKITLAGKTMFIDKTASGVSTLINEDAAAAKKSTANPLASIDSALSKVDAVRSSLGAIQNRFDSAITNLGNTVTNLNSARSRIEDADYATEVSNMSKAQILQQAGTSVLAQANQVPQNVLSLLRGGGGSAMVRSPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 643 aa, Artificial Sequence
  hIgG4-Fc-Paflagellin  fusion protein (with linker 1)
MYRMQLLSCIALSLALVTNSISMALTVNTNIASLNTQRNQNNSSASLNTSLQRLSTGSRINSAKDDAAGLQIANRLTSQVNGLNVATKNANDGISLAQTAEGALQQSTNILQRMRDLSLQSANGSNSDSERTALNGEVKQLQKELDRISNTTTFGGRKLLDGSFGVASFQVGSAANEIISVGIDEMSAESLNGTYFKADGGGAVTAATASGTVDIAIDITGGSAVNVKVDMKGNETAEQAAAKIAAAVNDANVGIGAFTDGAQISYVSKASADGTTSAVSGVAITDTGSTGAGTAAGTTTFTEANDTVAKIDISTAKGAQSAVLVIDEAIKQIDAQRADLGAVQNRFDNTINNLKNIGENVSAARGRIEDTDFAAETANLTKNQVLQQAGTAILAQANQLPQSVLSLLRGGGGSAMVRSPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - protein, 806 aa, Artificial Sequence
  hIgG4-Fc-Sfflagellin fusion protein (with linker 1)
MYRMQLLSCIALSLALVTNSISMAQVINTNSLSLITQNNINKNQSALSSSIERLSSGLRINSAKDDAAGQAIANRFTSNIKGLTQAARNANDGISVAQTTEGALSEINNNLQRIRELTVQASTGTNSDSDLDSIQDEIKSRLDEIDRVSGQTQFNGVNVLAKDGSMKIQVGANDGQTITIDLKKIDSDTLGLNGFNVNGGGAVANTAASKADLVAANATVVGNKYTVSAGYDAAKASDLLAGVSDGDTVQATINNGFGTAASATNYKYDSASKSYSFDTTTASAADVQKYLTPGVGDTAKGTITIDGSAQDVQISSDGKITASNGDKLYIDTTGRLTKNGSGASLTEASLSTLAANNTKATTIDIGGTSISFTGNSTTPDTITYSVTGAKVDQAAFDKAVSTSGNNVDFTTAGYSVNGTTGAVTKGVDSVYVDNNEALTTSDTVDFYLQDDGSVTNGSGKAVYKDADGKLTTDAETKAATTADPLKALDEAISSIDKFRSSLGAVQNRLDSAVTNLNNTTTNLSEAQSRIQDADYATEVSNMSKAQIIQQAGNSVLAKANQVPQQVLSLLQGGGGGSAMVRSPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - protein, 754 aa, Artificial Sequence
  hIgG4-Fc-Ecflagellin fusion protein (with linker 1)
MYRMQLLSCIALSLALVTNSISMAQVINTNSLSLITQNNINKNQSALSSSIERLSSGLRINSAKDDAAGQAIANRFTSNIKGLTQAARNANDGISVAQTTEGALSEINNNLQRVRELTVQATTGTNSESDLSSIQDEIKSRLDEIDRVSGQTQFNGVNVLAKNGSMKIQVGANDNQTITIDLKQIDAKTLGLDGFSVKNNDTVTTSAPVTAFGATTTNNIKLTGITLSTEAATDTGGTNPASIEGVYTDNGNDYYAKITGGDNDGKYYAVTVANDGTVTMATGATANATVTDANTTKATTITSGGTPVQIDNTAGSATANLGAVSLVKLQDSKGNDTDTYALKDTNGNLYAADVNETTGAVSVKTITYTDSSGAASSPTAVKLGGDDGKTEVVDIDGKTYDSADLNGGNLQTGLTAGGEALTAVANGKTTDPLKALDDAIASVDKFRSSLGAVQNRLDSAVTNLNNTTTNLSEAQSRIQDADYATEVSNMSKAQIIQQAGNSVLAKANQVPQQVLSLLQGGGGGSAMVRSPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 17 - DNA, 1596 bp, Artificial Sequence
  polynucleotide encoding hIgG4-Fc-Bsflagellin
atgtacaggatgcaactcctgtcttgcattgcactaagtcttgcacttgtcacgaattcgatatcgatgagaattaaccacaatattgcagcacttaacacattgaatcgtttgggttcaaacaacggagcagcacaaaagaatatggagaagctttcttcaggtcttcgtatcaaccgtgcgggagatgacgcagcaggtctagcgatctctgaaaaaatgagaggacaaatcagaggtcttgaaatggcttctaaaaactctcaagatggaatctctcttatccaaacagctgagggtgcattaactgaaactcatgcaattcttcaacgtatgcgtgaacttactgttcaagcaggaaacacaggtacacaacaagctgaagatcttggtgcaattaaagatgaaatggatgcgcttatcgaggaaattgatggcatttcaaaccgtactgaatttaacggtaaaaagttgctagacggaactaattctactgatggtttcacattccaaattggtgcgaatgctggccaacaactaaatgtaaaaattgacagcatgtcatcaactgctttaggagtaaacgcacttgatgtaacagatttcgctgctactgcttttgatgatcaacttaaaagtattgatactgccatcaatactgtatcaactcaacgtgctaaattaggtgcggtacaaaaccgtctagagcatacaatcaacaacttaggcgcttctggtgaaaacctgacagctgctgagtctcgtatccgtgacgttgacatggctaaagaaatgagcgagttcactaagaacaacattctttctcaagcttctcaagctatgcttgctcaagctaaccaacagcctcaaaacgtacttcaattattacgtggtggcggagggtctgccatggttagatctcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaataa

SEQ ID NO: 18 - DNA, 1524 bp, Artificial Sequence
  polynucleotide encoding mIgG1-Fc-Bsflagellin (with linker 1)
atgagaattaaccacaatattgcagcacttaacacattgaatcgtttgggttcaaacaacggagcagcacaaaagaatatggagaagctttcttcaggtcttcgtatcaaccgtgcgggagatgacgcagcaggtctagcgatctctgaaaaaatgagaggacaaatcagaggtcttgaaatggcttctaaaaactctcaagatggaatctctcttatccaaacagctgagggtgcattaactgaaactcatgcaattcttcaacgtatgcgtgaacttactgttcaagcaggaaacacaggtacacaacaagctgaagatcttggtgcaattaaagatgaaatggatgcgcttatcgaggaaattgatggcatttcaaaccgtactgaatttaacggtaaaaagttgctagacggaactaattctactgatggtttcacattccaaattggtgcgaatgctggccaacaactaaatgtaaaaattgacagcatgtcatcaactgctttaggagtaaacgcacttgatgtaacagatttcgctgctactgcttttgatgatcaacttaaaagtattgatactgccatcaatactgtatcaactcaacgtgctaaattaggtgcggtacaaaaccgtctagagcatacaatcaacaacttaggcgcttctggtgaaaacctgacagctgctgagtctcgtatccgtgacgttgacatggctaaagaaatgagcgagttcactaagaacaacattctttctcaagcttctcaagctatgcttgctcaagctaaccaacagcctcaaaacgtacttcaattattacgtggtggcggagggtctaccatggttagatctggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 19 - DNA, 1554 bp, Artificial Sequence
  polynucleotide encoding mIgG1-Fc-Bsflagellin (with linker 2)
atgagaattaaccacaatattgcagcacttaacacattgaatcgtttgggttcaaacaacggagcagcacaaaagaatatggagaagctttcttcaggtcttcgtatcaaccgtgcgggagatgacgcagcaggtctagcgatctctgaaaaaatgagaggacaaatcagaggtcttgaaatggcttctaaaaactctcaagatggaatctctcttatccaaacagctgagggtgcattaactgaaactcatgcaattcttcaacgtatgcgtgaacttactgttcaagcaggaaacacaggtacacaacaagctgaagatcttggtgcaattaaagatgaaatggatgcgcttatcgaggaaattgatggcatttcaaaccgtactgaatttaacggtaaaaagttgctagacggaactaattctactgatggtttcacattccaaattggtgcgaatgctggccaacaactaaatgtaaaaattgacagcatgtcatcaactgctttaggagtaaacgcacttgatgtaacagatttcgctgctactgcttttgatgatcaacttaaaagtattgatactgccatcaatactgtatcaactcaacgtgctaaattaggtgcggtacaaaaccgtctagagcatacaatcaacaacttaggcgcttctggtgaaaacctgacagctgctgagtctcgtatccgtgacgttgacatggctaaagaaatgagcgagttcactaagaacaacattctttctcaagcttctcaagctatgcttgctcaagctaaccaacagcctcaaaacgtacttcaattattacgtggtggcggagggtctggtggcggagggtctggtggcggagggtctaccatggttagatctggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 20 - DNA, 2286 bp, Artificial Sequence
  polynucleotide encoding hIgG4-Fc-Sdflagellin (with linker 1)
atgtacaggatgcaactcctgtcttgcattgcactaagtcttgcacttgtcacgaattcgatatcgatggcacaagtcattaatacaaacagcctgtcgctgttgacccagaataacctgaacaaatctcagtcctcactgagttccgctattgagcgtctgtcctctggtctgcgtatcaacagcgcgaaagacgatgcggcaggccaggcgattgctaaccgcttcacttctaatatcaaaggtctgactcaggcttcccgtaacgctaacgacggcatttctattgcgcagaccactgaaggtgcgctgaatgaaatcaacaacaacctgcagcgtgtgcgtgagttgtctgttcaggccactaacgggactaactctgattccgatctgaaatctatccaggatgaaattcagcaacgtctggaagaaatcgatcgcgtttctaatcagactcaatttaacggtgttaaagtcctgtctcaggacaaccagatgaaaatccaggttggtgctaacgatggtgaaaccattaccatcgatctgcaaaaaattgatgtgaaaagccttggccttgatgggttcaatgttaatgggccaaaagaagcgacagtgggtgatctgaaatccagcttcaagaatgttacgggttacgacacctatgcagcgggtgccgataaatatcgtgtagatattaattccggtgctgtagtgactgatgcagtagcaccggataaagtatatgtaaatgcagcaaacggtcagttaacaactgacgatgcggaaaataacactgcggttgatctctttaagaccactaaatctactgctggtaccgctgaagccaaagcgatagctggtgccattaaaggtggtaaggaaggagatacctttgattataaaggcgtgacttttactattgatacaaaaactggtgatgacggtaatggtaaggtttctactaccatcaatggtgaaaaagttacgttaactgtcgctgatattgccattggcgcggcggatgttaatgctgctaccttacaatcaagcaaaaatgtttatacatctgtagtgaacggtcagtttacttttgatgataaaaccaaaaacgagagtgcgaaactttctgatttggaagcaaacaatgctgttaagggcgaaagtaaaattacagtaaatggggctgaatatactgctaacgccacgggtgataagatcaccttagctggcaaaaccatgtttattgataaaacagcttctggcgtaagtacattaatcaatgaagacgctgccgcagccaagaaaagtaccgctaacccactggcttcaattgattctgcattgtcaaaagtggacgcagttcgttcttctctgggggcaattcaaaaccgttttgattcagccattaccaaccttggcaatacggtaaccaatctgaactccgcgcgtagccgtatcgaagatgctgactatgcaacggaagtttctaatatgtctaaagcgcagattctgcagcaggctggtacttccgttctggcgcaggctaaccaggttccgcaaaacgtcctctctttactgcgtggtggcggagggtctgccatggttagatctcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaataa

SEQ ID NO: 21 - DNA, 1932 bp, Artificial Sequence
  polynucleotide encoding hIgG4-Fc-Paflagellin (with linker 1)
atgtacaggatgcaactcctgtcttgcattgcactaagtcttgcacttgtcacgaattcgatatcgatggccttgaccgtcaacaccaacatcgcttcgctgaacactcagcggaaccagaacaactcttccgcgtcgctgaacacttcgctgcagcgtctgtccaccggttcgcgcatcaacagcgccaaggacgacgccgccggcctgcagatcgccaaccgtctgaccagccaggtcaacggcctgaacgtggctaccaagaacgccaacgatggtatctccctggcgcagaccgctgaaggcgccctgcagcagtcgaccaacatcctgcagcgtatgcgtgacctgtccctgcagtcggccaacggctccaacagcgactccgagcgtaccgctctgaatggcgaagtgaagcaactgcagaaagaactggatcgtatcagcaacaccaccaccttcggtggccgcaagctgctcgacggttccttcggcgtcgccagcttccaggtgggttcggccgccaacgaaatcatcagcgtcggcatcgacgagatgagcgcagagtcgctgaacggcacctacttcaaggctgacggcggcggcgcggtcactgctgcaaccgcttcgggcaccgtcgacatcgcgatcgacatcaccggcggcagcgccgtgaacgtcaaggtcgacatgaagggcaacgaaaccgccgagcaggcggctgccaagatcgccgctgcggtcaacgacgccaacgtcggcatcggtgccttcaccgacggcgcgcagatcagctacgtgtccaaggctagtgcggatggtactaccagtgcggtttccggcgtagccatcaccgacaccggcagcaccggcgcaggcaccgcggcaggcaccacgacgttcaccgaggctaacgacaccgtcgccaagatcgacatctccaccgcgaagggcgctcagtccgccgtgctggtgatcgacgaggcgatcaagcagatcgacgcccagcgtgccgacctcggtgcggtgcagaaccgcttcgacaacaccataaacaacctgaagaacatcggtgagaacgtatcggctgctcgcggccggatcgaagacaccgacttcgcagccgaaaccgccaacctgaccaagaaccaagtgctgcaacaagccggcaccgcgatcctggcccaggccaaccagctgccgcagtcggttctgagcctgctgcgcggtggcggagggtctgccatggttagatctcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaataa

SEQ ID NO: 22 - DNA, 2421 bp, Artificial Sequence
  polynucleotide encoding hIgG4-Fc-Sfflagellin (with linker 1)
atgtacaggatgcaactcctgtcttgcattgcactaagtcttgcacttgtcacgaattcgatatcgatggcacaagtcattaataccaacagcctctcgctgatcactcaaaataatatcaacaagaaccagtctgcgctgtcgagttctatcgagcgtctgtcttctggcttgcgtattaacagcgcgaaggatgacgccgcgggtcaggcgattgctaaccgttttacttctaacattaaaggcctgactcaggctgcacgtaacgccaacgacggtatttctgttgcacagaccactgaaggcgcgctgtccgaaatcaacaacaacttacagcgtatccgtgagctgacggttcaggcttctaccgggactaactctgattcggatctggactccattcaggacgaaatcaaatcccgtctcgacgaaattgaccgcgtatccggtcagacccagttcaacggcgtgaacgtactggcaaaagacggttcgatgaaaattcaggttggtgcgaatgacggccagactatcactattgatctgaagaaaattgactctgatacgctggggctgaatgggtttaatgtgaatggtggcggggctgtggctaatactgctgcatctaaagctgacttggtagctgctaatgcaacagtggtcggcaacaaatatactgtgagtgcgggttacgatgctgctaaagcgtctgatttgctggctggagttagtgatggtgatactgttcaggcaaccattaataacggcttcggaacggcggctagtgcaacgaattacaagtatgacagtgcaagtaagtcatactcttttgataccacaacggcttcagctgccgatgttcagaaatatttgaccccgggagttggtgataccgctaagggcactattactatcgatggttctgcacaggatgttcagatcagcagtgatggtaaaattacggcaagcaatggagataaactctacattgatacaactgggcgcttaacgaaaaacggctctggtgcttctttgactgaggctagtctgtccacacttgcagccaataataccaaagcgacaaccattgacattggcggtacctctatctcctttaccggtaatagtactacgccggacactattacttattcagtaacaggtgcaaaagttgatcaggcagctttcgataaagctgtatcaacctcgggaaacaatgttgatttcactactgcaggttatagcgtcaacggcacaactggcgctgtaacaaaaggtgttgattcggtttatgttgataacaacgaggcgttgaccacatctgatactgtagatttttacctacaggatgatggttcagtgactaacggcagcggtaaggcagtttataaagatgctgatggtaaattgacgacagatgctgaaacaaaagcagctacgacagccgatcccctgaaagctctggatgaagccatcagctccatcgacaaattccgctcctccctgggtgcggtgcaaaaccgtttggattccgcagtcaccaacctgaacaacaccactaccaacctgtctgaagcgcagtcccgtattcaggacgccgactatgcgaccgaagtgtccaacatgtcgaaagcgcagattatccagcaggccggtaactccgtgctggcaaaagccaaccaggtaccgcagcaggttctgtctctgctgcagggtggtggcggagggtctgccatggttagatctcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaataa

SEQ ID NO: 23 - DNA, 2265 bp, Artificial Sequence
  polynucleotide encoding hIgG4-Fc-Ecflagellin (with linker 1)
atgtacaggatgcaactcctgtcttgcattgcactaagtcttgcacttgtcacgaattcgatatcgatggcacaagtcattaataccaacagcctctcgctgatcactcaaaataatatcaacaagaaccagtctgcgctgtcgagttctatcgagcgtctgtcttctggcttgcgtattaacagcgcgaaggatgacgcagcgggtcaggcgattgctaaccgtttcacctctaacattaaaggcctgactcaggcggcccgtaacgccaacgacggtatctccgttgcgcagaccaccgaaggcgcgctgtccgaaatcaacaacaacttacagcgtgtgcgtgaactgacggtacaggccactaccggtactaactctgagtctgatctgtcttctatccaggacgaaattaaatcccgtctggatgaaattgaccgcgtatctggtcagacccagttcaacggcgtgaacgtgctggcaaaaaatggctccatgaaaatccaggttggcgcaaatgataaccagactatcactatcgatctgaagcagattgatgctaaaactcttggccttgatggttttagcgttaaaaataacgatacagttaccactagtgctccagtaactgcttttggtgctaccaccacaaacaatattaaacttactggaattaccctttctacggaagcagccactgatactggcggaactaacccagcttcaattgagggtgtttatactgataatggtaatgattactatgcgaaaatcaccggtggtgataacgatgggaagtattacgcagtaacagttgctaatgatggtacagtgacaatggcgactggagcaacggcaaatgcaactgtaactgatgcaaatactactaaagctacaactatcacttcaggcggtacacctgttcagattgataatactgcaggttccgcaactgccaaccttggtgctgttagcttagtaaaactgcaggattccaagggtaatgataccgatacatatgcgcttaaagatacaaatggcaatctttacgctgcggatgtgaatgaaactactggtgctgtttctgttaaaactattacctatactgactcttccggtgccgccagttctccaaccgcggtcaaactgggcggagatgatggcaaaacagaagtggtcgatattgatggtaaaacatacgattctgccgatttaaatggcggtaatctgcaaacaggtttgactgctggtggtgaggctctgactgctgttgcaaatggtaaaaccacggatccgctgaaagcgctggacgatgctatcgcatctgtagacaaattccgttcttccctcggtgcggtgcaaaaccgtctggattccgcggttaccaacctgaacaacaccactaccaacctgtctgaagcgcagtcccgtattcaggacgccgactatgcgaccgaagtgtccaatatgtcgaaagcgcagatcatccagcaggccggtaactccgtgttggcaaaagctaaccaggtaccgcagcaggttctgtctctgctgcagggtggtggcggagggtctgccatggttagatctcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaataa

SEQ ID NO: 24 - DNA, 43 bp, Artificial Sequence
  Ecflagellin-hIgG4-Fc2-5′ EcoRV-G
ccggatatcgatggcacaagtcattaataccaacagcctctcg

SEQ ID NO: 25 - DNA, 48 bp, Artificial Sequence
  Ecflagellin-(GGGGS)-hIgG1-Fc2-3′ NcoI-C
gaccatggcagaccctccgccaccctgcagcagagacagaacctgctg

SEQ ID NO: 26 - DNA, 43 bp, Artificial Sequence
  Paflagellin-hIgG4-Fc2-5′ EcoRV-G
ccggatatcgatggccttgaccgtcaacaccaacatcgcttcg

SEQ ID NO: 27 - DNA, 51 bp, Artificial Sequence
  Ecflagellin-(GGGGS)-hIgG1-Fc2-3′ NcoI-C
gaccatggcagaccctccgccaccgcgcagcaggctcagaaccgactgcgg

SEQ ID NO: 28 - DNA, 37 bp, Artificial Sequence
  Sdflagellin-hIgG4-Fc2-5′ EcoRV-G
ccggatatcgatggcacaagtcattaatacaaacagc

SEQ ID NO: 29 - DNA, 48 bp, Artificial Sequence
  Sdflagellin-(GGGGS)-hIgG1-Fc2-3′ NcoI-C
gaccatggcagaccctccgccaccacgcagtaaagagaggacgttttg

SEQ ID NO: 30 - DNA, 37 bp, Artificial Sequence
  Sfflagellin-hIgG4-Fc2-5′ EcoRV-G
ccggatatcgatggcacaagtcattaataccaacagc

SEQ ID NO: 31 - DNA, 45 bp, Artificial Sequence
  Sfflagellin-(GGGGS)-hIgG1-Fc2-3′ NcoI-C
gaccatggcagaccctccgccaccaccctgcagcagagacagaac

SEQ ID NO: 32 - DNA, 43 bp, Artificial Sequence
  CTGBsflagellin-hIgG4-Fc2-5′ EcoRV-G
ccggatatcgatgagaattaaccacaatattgcagcacttaac

SEQ ID NO: 33 - DNA, 54 bp, Artificial Sequence
  Bsflagellin-(GGGGS)-hIgG1-Fc2-3′ NcoI-C
gaccatggcagaccctccgccaccacgtaataattgaagtacgttttgaggctg

SEQ ID NO: 34 - DNA, 42 bp, Artificial Sequence
  pET49b-bsflagellin vector forward primer
ccggaattcatgagaattaaccacaatattgcagcacttaac

SEQ ID NO: 35 - DNA, 54 bp, Artificial Sequence
  pET49b-bsflagellin vector reverse primer - 1
gaccatggtagaccctccgccaccacgtaataattgaagtacgttttgaggctg

SEQ ID NO: 36 - DNA, 84 bp, Artificial Sequence
  pET49b-bsflagellin vector reverse primer - 2
gaccatggtagaccctccgccaccagaccctccgccaccagaccctccgccaccacgtaataattgaagtacgttttgaggctg

(19) United States
(12) Patent Application Publication
(10) Pub. No.: US 2022/0194993 A1
(43) Pub. Date: Jun. 23, 2022

(54) FLAGELLIN FUSION PROTEIN AND USE THEREOF

(71) Applicant: INDUSTRY FOUNDATION OF CHONNAM NATIONAL UNIVERSITY, Gwangju (KR)

(72) Inventors: Kyung A CHO, Gwangju (KR); Jae Sung LIM, Gwangju (KR)

(21) Appl. No.: 17/605,455

(22) PCT Filed: Apr. 22, 2020
(86) PCT No.: PCT/KR2020/005327

§ 371 Date: Oct. 21, 2021

(30) Foreign Application Priority Data
Apr. 22, 2020 (KR) 10-2019-0046866

(51) Int. Cl. C07K 14/195; A61K 39/39

(52) U.S. Cl. CPC C07K 14/195; A61K 2039/55516; A61K 39/39

(57) ABSTRACT

The present invention relates to a flagellin fusion proteins and a use thereof and, more specifically, to a fusion protein comprising flagellin, a fragment thereof; or a variant thereof; and an immunoglobulin Fc region and use in which a toll-like receptor 5 (TLR5) stimulating activity thereof is used. The fusion protein provided by the present invention has remarkably excellent toll-like receptor 5 (TLR5) pathway activation ability compared to wild-type flagellin, a fragment thereof, or a variant thereof, and therefore can be greatly utilized to develop a therapeutic agent and/or a vaccine adjuvant for a disease that can be prevented, improved, or treated through activation of the TLR5 pathway.

### TECHNICAL FIELD

This application claims priority to Korean Patent Application No. 10-2019-0046866 filed on Apr. 22, 2019, and the entire specifications of which are incorporated herein by reference in their entireties.

The present invention relates to a flagellin fusion proteins and a use thereof and, more specifically, to a fusion protein comprising flagellin, a fragment thereof; or a variant thereof; and an immunoglobulin Fc region and use in which a toll-like receptor 5 (TLR5) stimulating activity thereof is used.

### BACKGROUND OF THE INVENTION

Flagella are an important component that determines the motility of bacteria and is largely composed of a hook, a basal body, and a filament. It is also known that flagella have a function of determining bacterial swimming or swarming motility, bacterial taxis, and forming a biofilm to determine the ability of pathogenic microorganisms to adhere. The structural unit protein constituting the filament of the flagella is called flagellin, and the flagellin is regularly assembled to form the filament. Hayashi et al. reported that mammalian-expressed TLR5 recognizes flagellin from Gram-negative and Gram-positive bacteria and activates NF-κB (Hayashi F, Smith K D, Ozinsky A, Hawn T R, Yi E C, Goodlett D R, Eng J K, Akira S, Underhill D M, Aderem A: Nature 410:1099-1103, 2001).

Flagellin is a structural protein that assembles into the whip-stalk-like filaments of bacterial flagella, which extend from the cell surface and function to allow bacteria to move. Flagellin promotes the penetration and invasion of pathogenic bacteria into host cells by acting as a virulence factor. Because flagellin is found exclusively in bacteria and is one of the most abundant proteins in flagellated bacteria, flagellin is a major target for host immune surveillance. Upon bacterial invasion, flagellin activates innate immunity, which is detected by Toll-like receptor 5 (TLR5) and NAIP5/NLRC4 in the host and contributes to the immediate clearance of pathogens from the host.

TLR5 is an innate immune receptor located on the cell surface and consists of an extracellular leucine-rich repeat (LRR) domain, a transmembrane domain and an intracellular domain. TLR5 recognizes flagellin as a pathogen-associated molecular pattern using an extracellular domain and activates the MyD88-dependent signaling pathway and NF-κB-mediated inflammatory cytokine production.

Flagellin has been of interest as a target for the development of vaccine carrier proteins or vaccine adjuvants because it serves as the first line of defense against flagellate-pathogenic bacteria. A fusion protein of antigen and flagellin has been demonstrated to be effective as an experimental vaccine against a variety of communicable diseases including West Nile fever, malaria, infectious diseases and tuberculosis, and it has been reported that flagellin-induced TLR5 activation also protects hematopoietic cells and radiation-induced gastrointestinal tissue and affects the survival and growth of cancer cells.

Flagellin contains two to four domains. For example, the Bacillus subtilis Hag flagella, Pseudomonas aeruginosa type A FliC flagella, Salmonella enterica subspecies enterica serovar Typhimurium FliC flagellin each contain 2 (D0 and D1), 3 (D0, D1, and D2) and 4 (D0, D1, D2, and D3) domains. The D0 and D1 domains common in these are located at the center of flagellar filaments by mediating inter-flagellar interactions and are highly conserved among bacterial species due to the functional importance of filament formation. Since flagellin monomer, not a polymerized filament, activates TLR5, it is thought that the aforementioned D0 and D1 domains may be major stimulators of TLR5. In the 3- and 4-domain flagellins, the D1 domain extends to auxiliary domains (D2 and D3) on the surface of the flagellar filaments, contributing little or no to filament formation. Unlike the D0 and D1 domains, the D2 or D3 domains exhibit substantial changes in sequence and structure, and are believed to activate adaptive immunity and induce undesirable toxicity in flagellin-based therapies. Therefore, the radiation therapy bio-drug CBLB502 containing D0/D1 was developed by removing the hypervariable region (D2 and D3 domains) from Salmonella flagellin.

Many Gram-positive bacteria, such as Bacillus subtilis and Clostridium difficile, express flagellin deficient in the hypervariable region, so flagellar filaments contain the minimum regions (D0 and D1 regions) required for TLR5 activation and flagellin polymerization.

Flagellin-TLR5 interaction and its cellular consequences have been extensively studied using Salmonella flagellin. Structural and biochemical studies of the complex between Salmonella enterica subspecies enterica serovar Dublin flagellin D1-D2 region (sdflagellin D1-D2) and the N-terminal fragment of zebrafish TLR5 is known that flagellin and TLR5 form a 1:1 complex through ‘primary bond’, afterwards, to be homodimerized into a 2:2 complex through ‘second dimerization’.

Based on the results of many studies on the interaction between flagellin and TLR5, various studies are being conducted to enhance TLR5 activation through the modification of flagellin, but there is no research on a new type of flagellin protein targeting the flagellin-TLR5 2:2 complex structure.

### DETAILED DESCRIPTION OF THE INVENTION

### Technical Problem

Accordingly, the present inventors focused on the formation of a 2:2 complex structure when flagellin activates TLR5, and as a result of repeated studies to develop a new type of protein with improved TLR5 activation ability, it was discovered that a new type of fusion protein in which flagellin and immunoglobulin Fc are fused exhibits significantly superior TLR5 activation ability compared to wild-type flagellin, known flagellin fragments, and the like, and completed the present invention.

Accordingly, an object of the present invention is to provide a fusion protein comprising a flagellin, a fragment thereof, or a variant thereof; and an immunoglobulin Fc region.

Another object of the present invention is to provide a polynucleotide encoding the fusion protein.

Another object of the present invention is to provide a vector comprising the polynucleotide.

Another object of the present invention is to provide a transformant transformed with the vector.

Another object of the present invention is to provide a pharmaceutical composition comprising the fusion protein as an active ingredient.

It is also to provide a pharmaceutical composition consisting of the fusion protein.

It is also to provide a pharmaceutical composition essentially consisting of the fusion protein.

Another object of the present invention is to provide a vaccine adjuvant comprising the fusion protein as an active ingredient.

It is also to provide a vaccine adjuvant consisting of the fusion protein.

It is also to provide a vaccine adjuvant essentially consisting of the fusion protein.

Another object of the present invention is to provide use of the fusion protein for the preparation of a pharmaceutical agent for the treatment of injury by radiation exposure; the treatment of reperfusion injury; the treatment of inflammatory bowel disease; the treatment of autoimmune disease; the treatment of viral infection; the treatment of metabolic disease; the treatment of aging; the enhancement of immune function; or the treatment of cancer.

Another object of the present invention is to provide a method for treating injury by exposure to radiation; treating reperfusion injury; treating inflammatory bowel disease; treating autoimmune disease, treating viral infection; treating metabolic disease; treating aging; enhancing immune function; or treating cancer, comprising administering to a subject in need thereof an effective amount of a composition comprising the fusion protein as an active ingredient.

### Technical Solution

In order to achieve the above object of the present invention, the present invention provides a flagellin, a fragment thereof or a variant thereof; and an immunoglobulin Fc region.

In order to achieve another object of the present invention, the present invention provides a polynucleotide encoding the fusion protein.

In order to achieve another object of the present invention, the present invention provides a vector comprising the polynucleotide

In order to achieve another object of the present invention, the present invention provides a transformant transformed with the vector

In order to achieve another object of the present invention, the present invention provides a pharmaceutical composition comprising the fusion protein as an active ingredient.

The present invention also provides a pharmaceutical composition consisting of the fusion protein.

The present invention also provides a pharmaceutical composition essentially consisting of the fusion protein.

In order to achieve another object of the present invention, the present invention provides a vaccine adjuvant comprising the fusion protein as an active ingredient.

The present invention also provides a vaccine adjuvant consisting of the fusion protein.

The present invention also provides a vaccine adjuvant essentially consisting of the fusion protein.

In order to achieve another object of the present invention, the present invention provides use of the fusion protein for the preparation of a pharmaceutical agent for the treatment of injury by radiation exposure; the treatment of reperfusion injury; the treatment of inflammatory bowel disease; the treatment of autoimmune disease; the treatment of viral infection; the treatment of metabolic disease; the treatment of aging; the enhancement of immune function; or the treatment of cancer.

In order to achieve another object of the present invention, the present invention provides a method for treating injury by exposure to radiation; treating reperfusion injury; treating inflammatory bowel disease; treating autoimmune disease, treating viral infection; treating metabolic disease; treating aging; enhancing immune function; or treating cancer, comprising administering to a subject in need thereof an effective amount of a composition comprising the fusion protein as an active ingredient.

Hereinafter, the present invention will be described in detail.

The present invention provides a flagellin, a fragment thereof or a variant thereof; and an immunoglobulin Fc region.

In the present invention, the flagellin may induce an immune response in an infected host when the flagellate bacterium is infected. More specifically, Toll-like receptor 5 (TLR5) present on the cell membrane surface of the human body induces intracellular signal transduction through interaction with the flagellin, and through this, the expression of NF-kB, a transcription factor, is increased to induce activation of innate immune signals, as well as regulate acquired immune responses.

Flagellin proteins are described well, for example, in U.S. Pat. Nos. 6,585,980, 6,130,082; 5,888,810; 5,618,533; 4,886,748 and US Patent Publication No. US2003/0044429 A1; and Donnelly et al. (2002) J. Biol. Chem. 43:4045 and the like. Most Gram-negative bacteria express flagella, a surface structure that provides motility. A flagellum consists of a basal body, a filament, and a hook connecting the two. The filaments consist of a long polymer of flagellin, a single protein, with a small cap protein formed at the tip.

Polymerization of flagellin is mediated by regions conserved at the N- and C-terminus, whereas the intermediate hypervariable region of flagellin protein is highly variable in sequence and length between species.

In the present invention, the flagellin may be flagellin derived from any suitable bacteria. A number of flagellin genes have been cloned and sequenced in the art and may be referred to.

In the present invention, the non-limiting source of the flagellin is Bacillus genus, Salmonella genus Helicobacter genus, Vibrio genus, Serratia genus, Shigella genus, Treponema genus, Legionella genus, Borrelia genus, Clostridium genus, Agrobacterium genus, Bartonella genus, Proteus genus, Pseudomonas genus, Escherichia genus, Listeria genus, Yersinia genus, Campylobacter genus, Roseburia genus or Marinobacter genus microorganisms, preferably is Bacillus genus, Salmonella genus or Vibrio genus microorganisms.

More preferably, in the present invention, the flagellin may be derived from Salmonella enteritidis, Salmonella typhimurium, Salmonella Dublin, Salmonella enterica, Helicobacter pylori, Vibrio cholera, Vibrio vulnificus, Vibrio fibrisolvens, Serratia marcesens, Shigella flexneri, Treponema pallidum, Borrelia burgdorferei, Clostridium difficile, Agrobacterium tumefaciens, Bartonella clarridgeiae, Proteus mirabilis, Bacillus subtilis, Bacillus cereus, Bacillus halodurans, Pseudomonas aeruginosa, Escherichia coli, Listeria monocytogenes, Yersinia pestis, Campylobacter spp, Roseburia spp or Marinobacter spp.

Even more preferably, in the present invention, the flagellin may be derived from Salmonella enteritidis, Salmonella typhimurium, Salmonella Dublin, Salmonella enterica, Vibrio cholera, Vibrio vulnificus, Vibrio fibrisolvens, Bacillus subtilis, Bacillus cereus or Bacillus halodurans,

Most preferably, it may be flagellin derived from Bacillus subtilis.

TLR5 recognizes the evolutionarily conserved region of bacterial flagellin required for flagellar filament assembly and movement, but some a and E Proteobacteria (Campylobacter jejuni, Helicobacter pylori, and Bartonella bacilliformis) flagellins are not recognized by TLR5, as reported in the following papers. [(2003) Microbes Infect. 5, 1345-1356.; J. Infect. Dis. 189, 1914-1920.; (2002) Nat. Rev. Cancer 2, 28-37; (2001) Clin. Infect. Dis. 32, 1201-1206. pmid:11283810]. Bacterial flagellins recognized by TLR5 have similarly conserved major amino acid sequences in the D0 and D1 domains, and bacterial flagellins that are not recognized by TLR5 have different amino acid sequences (PNAS Jun. 28, 2005 102 (26) 9247-9252; Scientific Reports 7:40878 DOI: 10.1038/srep40878). Accordingly, in the present invention, the flagellin may be flagellin including a conserved sequence recognized by TLR5 in the D0 and D1 domains.

In one embodiment of the present invention, with reference to previously reported papers, flagellin of five representative bacteria in which a common amino acid sequence recognized by TLR5 is conserved in the D0 and D1 domains was prepared as an Fc-fusion protein.

The N-terminal and C-terminal constant regions of flagellin are well-characterized in the art, and for example, reference can be made to the literature[Mimori-Kiyosue et al., (1997) J. Mol. Byrol. 270: 222-237; lino et al., (1977) Ann. Genet. 11:161-182; and Schoenhals et al. (1993) J. Bacteriol. 175: 5395-5402]. As will be well understood by those skilled in the art, the size of the constant region may vary somewhat depending on the source of the flagellin protein. In general, an N-terminal constant domain comprises about 170 or 180 N-terminal amino acids of a protein, whereas a C-terminal constant domain typically comprises about 85-100 C-terminal amino acids. The central hypervariable region varies considerably depending on the size and order among bacteria, and most of the difference in molecular mass can be explained by the hypervariable region. The N- and C-terminal constant regions of flagellin proteins from various bacteria are known, and flagellin derived from bacteria, which is not yet known, can also be easily determined by a person skilled in the art using techniques known in the art to determine the crystal structure of flagellin monomer.

As used herein, the terms “flagellin”, “flagellin N-terminal constant region” and “flagellin C-terminal constant region” include flagellin active fragments and variants derived from any of the above exemplified bacteria. In addition, wild-type flagellin or portions of flagellin may be modified to increase safety and/or immune response and/or as a result of cloning procedures or other laboratory manipulations, and such modifications (or variants) are also included within the scope of the present invention.

In the present invention, the flagellin may include full-length flagellin, or an active fragment thereof. In addition, terms such as “flagellin”, “flagellin N-terminal constant region” and “flagellin C-terminal constant region” include naturally occurring amino acid sequences, or it may also comprise an amino acid sequence substantially identical to or similar to the amino acid sequence of a naturally occurring flagellin, flagellin N-terminal constant region, or flagellin C-terminal constant region, respectively.

In the present invention, an “active fragment” of flagellin, flagellin N-terminal constant region, flagellin C-terminal constant region, or any other portion of flagellin comprises at least about 50, 75, 100, 125, 150, 200, 250 or at least 300 contiguous amino acids and/or less than about 300, 250, 200, 150, 125, 100 or 75 amino acids of contiguous amino acids, and combinations of these may also be included as long as the lower limit is less than the upper limit. The active fragment may refer to a fragment capable of activating the TLR5 pathway in a host.

In certain embodiments, the active fragment is capable of activating the TLR5 pathway with at least about 50%, 75%, 80%, 85%, 90%, or 95% of full-length flagellin, or activates the TLR5 pathway to the same or essentially the same extent as the full-length flagellin or flagellin region, or activates the TLR5 pathway to a higher degree compared to full-length flagellin or flagellin region.

In the present invention, the active fragment may refer to at least one portion of flagellin exhibiting TLR5 pathway activity. The “at least one portion” may refer to a portion exhibiting TLR5 pathway activity in domains 0, 1, 2 and 3 of flagellin. More specifically, the active fragment may be a hypervariable region removed from full-length flagellin. The hypervariable region may vary depending on the type of bacteria from which flagellin is derived, and among the entire sequence of a specific flagellin, the sequence corresponding to the hypervariable region can be easily identified and removed by those skilled in the art. For example, N-terminal domains 0, 1, 2; domain 3; and domain 3, or domains 2 and 3 may be hypervariable regions in the case of full-length flagellin comprising C-terminal domains 2, 1, 0, and N-terminal domain 0, 1; domain 2; domain 2 may be a hypervariable region in the case of full-length flagellin including C-terminal domains 1 and 0. Alternatively, in the case of flagellin of a form not including a hypervariable region (For example, flagellin derived from many Gram-positive bacteria may not contain a hypervariable region.), the sequence of a hinge region in which folding of the flagellin protein occurs may be partially removed.

The term “hypervariable region” used in the present invention may be expressed as a propeller domain or region, a hinge, a hypervariable region, a variable domain or region, and the like.

In the present invention, the removal of the hypervariable region may mean that the entire domain corresponding to the hypervariable region may be removed, or a part of the sequence of the hypervariable region may be removed.

In the present invention, the active fragment may be flagellin in a form in which the hypervariable region of wild-type flagellin is removed and an artificial sequence (i.e., the hinge or linker of the artificial sequence) is inserted into the removed hypervariable region.

In the present invention, the flagellin fragment of the present invention may refer to a fragment exhibiting TLR5 pathway activity comprising at least one selected from the group consisting of C-terminal domain 0, C-terminal domain 1, C-terminal domain 2, N-terminal domain 2, N-terminal domain 1, N-terminal domain 0 of wild type flagellin and a region exhibiting at least 80% amino acid sequence homology with each of the domains.

In certain embodiments, the active fragment of flagellin is capable of activating the TLR5 pathway by at least about 50%, 75%, 80%, 85%, 90%, or 95% of full-length flagellin, or activating the TLR5 pathway to the same or essentially the same extent as the full-length flagellin or flagellin region, or higher activation of the TLR5 pathway compared to full-length flagellin or flagellin sites.

The present invention also includes proteins having the full-length sequence of wild-type flagellin as well as amino acid sequence variants thereof. In the present invention, the variant refers to a protein having a different sequence due to deletion, insertion, non-conservative or conservative substitutions, substitution of amino acid analogs or a combination thereof of some amino acid residues of a wild-type flagellin or a fragment thereof. Amino acid exchanges that do not entirely alter the activity of the molecule (ie, the ability to activate the TLR5 pathway) are known in the art (H. Neurath, R. L. Hill, The Proteins, Academic Press, New York, 1979).

In some cases, the variant of the present invention may be a full-length flagellin modified by phosphorylation, sulfation, acrylation, glycosylation, methylation, farnesylation or the like or a fragment thereof.

In certain embodiments, the variant of flagellin or fragment thereof is capable of activating the TLR5 pathway by at least about 50%, 75%, 80%, 85%, 90%, or 95% of full length flagellin or fragment thereof, or activating the TLR5 pathway to the same or essentially the same extent as full-length flagellin or a fragment thereof, or higher activation of the TLR5 pathway compared to full-length flagellin or a fragment thereof.

In the present invention, the flagellin, a fragment thereof, or a variant thereof may be in the form of a fusion protein comprising another polypeptide. For example, the flagellin may be a fusion protein comprising one or more antigens. Non-limiting examples of the antigen include S. pneumoniae PspA1 antigen, S. pneumoniae PspA2 antigen, S. pneumoniae PspA3 antigen, S. pneumoniae PspA4 antigen, S. pneumoniae PspA5 antigen and/or S. pneumoniae PspA6 antigen. Alternatively, for example, the flagellin may be in the form of a fusion protein to which one or more immunomodulatory substances are bound. The immunomodulatory substance may be included without limitation as long as it is known in the art to increase an immune response, and non-limiting examples thereof include interferon-α, interferon-β, interferon-γ, interferon-ω, interferon-τ, interleukin-1α, interleukin-1β, interleukin-2, interleukin-3, interleukin-4, interleukin-5, interleukin-6, interleukin-7, interleukin-8, interleukin-9, interleukin-10, interleukin-11, interleukin-12, interleukin-13, interleukin-14, interleukin-18, B cell growth factor, CD40 ligand, TNF-α, TNF-β, CCL25, CCL28 or an active fragment thereof.

As used herein, the term “percent (%) sequence homology” is defined as the percentage of amino acid residues in a candidate sequence that are identical with the amino acid residue in the reference polypeptide, after aligning the sequences and introducing gaps, without considering any conservative substitutions as part of the sequence identity to achieve the maximum percent sequence identity if necessary. Alignment for purposes of determining percent amino acid homology can be achieved, for example, using publicly available computer software programs and various methods that are within the skill in the art and using BLAST, blast-2, ALIGN, or Megalign (DNASTAR) software. One of skill in the art can determine appropriate parameters for alignment measurements, including any algorithms needed to achieve maximal alignment over the entire length of the sequences being compared. For the purposes herein, the percent (%) amino acid sequence homology of a given amino acid sequence B or of the given amino-acid sequence A to the given amino acid sequence B is calculated as follows: 100 times the fraction X/Y, where X is the number of amino acid residue scores that are identically matched by the sequence alignment program in the program alignment of A and B, and Y is the total number of the amino acid residues in B. It will be understood that if the length of amino acid sequences A is not equal to the length of amino acid sequence B, then the percent (%) amino acid sequence homology of A to B will not equal the percent (%) amino acids) sequence identity of B to A.

In certain embodiments, the flagellin, the fragment thereof, or the variant thereof may consist of an amino acid sequence selected from the group consisting of SEQ ID NOs: 1-5 or an amino acid sequence having at least 80% sequence homology thereto.

As used herein, the term “Fc region” refers to the C-terminal portion of an immunoglobulin chain constant region, and specifically refers to an immunoglobulin heavy chain constant region or a portion thereof.

The immunoglobulin Fc region of the present invention includes a native amino acid sequence as well as a mutant thereof. An amino acid sequence mutant means that one or more amino acid residues in a natural amino acid sequence have a different sequence by deletion, insertion, non-conservative or conservative substitution, or a combination thereof. For example, in the case of IgG Fc, amino acid residues 214 to 238, 297 to 299, 318 to 322, or 327 to 331 known to be important for binding may be used as suitable sites for modification. In addition, various types of mutants are possible, such as those in which a site capable of forming a disulfide bond is removed, some amino acids at the N-terminus of the native Fc are removed, or a methionine residue may be added to the N-terminus of the native Fc. In addition, in order to eliminate effector function, the complement binding site, e.g., the C1q binding site, may be removed, or the ADCC site may be removed. Techniques for preparing such an immunoglobulin Fc region sequence mutant are disclosed in International Patent Publication No. 97/34631 and International Patent Publication No. 96/32478 and the like.

Amino acid exchanges in proteins and peptides that do not entirely alter the activity of the molecule are known in the art. The most commonly occurring exchange is the exchange between the amino acid residues Ala/Ser, Val/Ile, Asp/Glu, Thr/Ser; Ala/Gly, Ala/Thr, Ser/Asn, Ala (Val), Ser/Gly (Thr/Phe), Ala/Pro, Lys/Arg (Asp/Asn), Leu/Iles, Leu/Val (Ala/Glu), Asp/Gly.

In some cases, it may be modified by phosphorylation, sulfation, acrylation, glycosylation, methylation, farnesylation, acetylation, amidation and the like.

The above-described Fc mutant is a mutant which exhibits the same biological activity as the Fc region of the present invention but has increased structural stability to heat, pH and the like of the Fc region.

In addition, such an Fc region may be obtained from natural forms isolated in vivo in animals such as humans and cattle, goats, pigs, mice, rabbits, hamsters, rats, guinea pigs and the like, or may be recombinant forms obtained from transformed animal cells or microorganisms or mutants thereof. Here, the method of obtaining from natural forms can be obtained by separating whole immunoglobulins from the body of a human or animal and then treating with protease. When treated with papain, it is cleaved into Fab and Fc, and when treated with pepsin, it is cleaved into pF′c and F(ab)2. In this case, Fc or pF′c may be separated using size-exclusion chromatography or the like.

In addition, the immunoglobulin Fc region may be a form in which a natural sugar chain, a sugar chain increased as compared with the natural form, a sugar chain decreased as compared with the natural form, or a sugar chain has been removed. Conventional methods such as chemical methods, enzymatic methods, and genetic engineering methods using microorganisms can be used to increase or eliminate the immunoglobulin Fc sugar chain. Here, the immunoglobulin Fc region from which the sugar chain has been removed from the Fc significantly reduces the binding ability of complement (c1q), reduces or eliminates antibody-dependent cytotoxicity or complement-dependent cellular cytotoxicity, and thus does not induce an unnecessary immune response in vivo. In this regard, a form more suitable for the original purpose of the drug carrier will be referred to as an immunoglobulin Fc region in which the sugar chain is removed or non-glycosylated.

In the present invention, the term “Deglycosylation” refers to an Fc region from which sugars have been removed by an enzyme, and “Aglycosylation” means a Fc region produced in prokaryotic animals, preferably Escherichia coli, and not glycosylated.

In addition, the immunoglobulin Fc region may be an Fc region derived from IgG, IgA, IgD, IgE, IgM or a combination thereof or a hybrid thereof. It may preferably be from IgG or IgM which is most abundant in human blood and most preferably from IgG which is known to enhance the half-life of the ligand binding protein.

On the other hand, in the present invention, “combination” means that when forming dimers or multimers, a polypeptide encoding a single chain immunoglobulin Fc region of the same origin forms a bond with a single chain polypeptide of different origin. That is, it is possible to prepare dimers or multimers from two or more fragments selected from the group consisting of Fc fragments of IgG Fc, IgA Fc, IgM Fc, IgD Fc and IgE.

“Hybrid” in the present invention is the term meaning that there is a sequence corresponding to an immunoglobulin Fc fragment of at least two different origins in a single immunoglobulin Fc region. Several types of hybrids are possible for the present invention. That is, a hybrid of domains of one to four domains from the group consisting of CH1, CH2, CH3 and CH4 of IgG Fc, IgM Fc, IgA Fc, IgE Fc and IgD Fc is possible and may comprise a hinge.

On the other hand, IgG can also be divided into subclasses of IgG1, IgG2, IgG3 and IgG4, and in the present invention, a combination thereof or hybridization thereof is also possible. It may preferably be derived from the Fc of human immunoglobulin IgG1.

In another embodiment, the immunoglobulin Fc region may comprise one or more selected from the group consisting of CH1, CH2, CH3 and CH4 domains of an immunoglobulin heavy chain constant region. For example, an immunoglobulin Fc region that may be used in the preparation of the fusion proteins of the present invention may comprise: (a) CH1 domain, CH2 domain, CH3 domain, and CH4 domain; (b) CH1, CH2, and CH3 domain; (c) CH1 domain, CH2 domain, and CH4 domain, (d) CH1 domain, CH3 domain, and CH4 domain; (e) CH2 domains, CH3 domain and CH4 domain: (f) CH1 domain and CH4 domain); (g) CH1 domain and CH3-domain; (h) CH1 domain and CH2 domain; (i) CH2 domain and CH4 domain; (j) CH2 domain and CH3 domain; (k) CH3 domain and CH4 domain; or a combination of two or more domains and an immunoglobulin hinge region. Most preferably, the immunoglobulin Fc region may consist of the CH2 and CH3 domain of a heavy chain constant region.

In addition to the human immunoglobulin Fc region, the present invention can also use other immunoglobulin Fc regions or fragment sequences thereof, including amino acid sequences encoded by nucleotide sequences disclosed in the GenBank and/or EMBL database, such as, for example, AF045536.1 (Macaca fascicularis), AF045537.1 (Macca mulatta), AB016710 (Felix catus), K00752 (Oryctolagus cuniculus), U03780 (Sus scrofa), Z48947 (Camelus Fromedarius), X62916 (Bos taurus), L07789 (Mustela vision), X6997 (Ovis aries), U17166 (Cricetulus migratorius), X07189 (Rattus ratus), AF57619.1 (Trichosurus vulpecula) or AF035195 (Monodelphis Domestica) and the like.

In the present invention, the fusion protein may be one in which the N-terminus or C-terminus of the flagellin, the fragment thereof, or the variant thereof is bound to the N-terminus or C-terminus of the immunoglobulin Fc region. Specifically, the N-terminus of the flagellin, the fragment thereof, or the variant thereof may be bound to the C-terminal of an immunoglobulin Fc region, or the C-terminus of the flagellin, the fragment thereof or the variant thereof is bound to an N-terminus of an immunoglobulins Fc region. Preferably, the C-terminus of the flagellin, the fragment thereof or the variant thereof may be bound to the N-terminal of the immunoglobulin Fc region.

In a particular embodiment, the immunoglobulin Fc region of the invention may consist of the amino acid sequence of SEQ ID NO: 6 or 7 or an amino acid sequence exhibiting at least 80% sequence homology thereto.

Meanwhile, in the present invention, each of the components constituting the fusion protein, i.e., the flagellin, the fragment thereof or the variant thereof, and the immunoglobulin Fc region may be directly connected to each other or connected via a linker. In general, the term “linker” refers to a nucleic acid, amino acid or non-peptide residue that can be inserted between one or more molecules, e.g., at least one component domain. For example, linkers can be used to provide the desired sites of interest between the components to facilitate manipulation. A linker may also be provided to enhance expression of the fusion protein from the transformant, and to reduce steric hindrance so that the component can assume its optimal tertiary structure and/or interact properly with the target molecule. The linker sequence may comprise one or more amino acids naturally linked to the receptor component, or may be an added sequence used to enhance expression of the fusion protein, to provide a desired site of specific interest, to allow the component domain to form an optimal tertiary structure, and/or to enhance interaction of the component with its target molecule.

Preferably, the linker can increase the flexibility of the fusion protein without interfering with the structure of each component in the fusion proteins. In some embodiments, the linker residue is a peptide linker having a length of 2 to 100 amino acids. Exemplary linkers include linear peptides having at least two amino acid residues, such as Gly-Gly, Gly-Ala-Gly; Gly-Pro-Ala; Gly (G)n; and Gly-Ser (GS) linkers. GS linkers described herein include, but are not limited to, (GS)n, (GSGSG)n, (G2S)n, G2S2G, (G2SG)n, (G3S)n, (G4S)n, (GGSGG)nGn, GSG4SG4SG and (GGGGS)n, where n is 1 or greater. One example of a (G)n linker includes a G9 linker, and examples of (GGGS)n linker include a GGGS or (GGGGS)3 linker. Suitable linear peptides include polyglycine, polyserine, polyproline, polyalanine and oligopeptides consisting of alanyl and/or serinyl and/or prolinyl and/least glycyl amino acid residues. The linker residues may be used to link the components of the fusion protein disclosed herein.

In the present invention, the linker may consist of the amino acid sequence of SEQ ID NO: 8 or 9.

The fusion proteins described herein may or may not comprise a signal peptide that functions to secrete the fusion protein from a host cell. The nucleic acid sequence encoding the signal peptide may be operably linked to the nucleic acid sequences encoding the protein of interest. In some embodiments, the fusion protein comprises a signal peptide. In some embodiments, the fusion protein does not comprise a signal peptide.

The fusion proteins described herein may also comprise modified forms of protein binding peptides. For example, the fusion protein component may have post-translational modifications including, for example, glycosylation, sialylation, acetylation and phosphorylation to any protein binding peptide.

Unless stated otherwise, the fusion proteins of the invention are administered as polypeptides (or nucleic acids encoding the polypeptides) that are themselves not part of a live, killed or recombinant bacterial or viral vectorized vaccine. Further, unless otherwise specified, the fusion proteins of the invention are isolated fusion proteins, e.g., not incorporated into flagella.

“Fusion” in the context of the present invention refers to the integration of two molecules of different or identical function or structure, which may be fusion by any physical, chemical or biological method with which the peptide may bind. The fusion protein or the polypeptide constituting the fusion protein can be produced by a chemical peptide synthesis method known in the art, or a gene encoding the fusion proteins can be amplified by PCR (polymerase chain reaction) or synthesized by a known method, and then cloned into an expression vector and expressed.

In certain embodiments of the invention the fusion protein may consist of an amino acid sequence selected from the group consisting of SEQ ID NOs: 10-16.

The invention also provides a polynucleotide comprising a nucleotide sequence encoding the fusion protein.

The polynucleotide is not particularly limited in the combination of bases constituting the polynucleotide as long as it can encode the polypeptide of the present invention. The polynucleotide can be provided as a nucleic acid molecule in the form of a single strand or a double strand, including both DNA, cDNA and RNA sequences.

Preferably, the polynucleotide of the present invention may have a nucleotide sequence selected from the group consisting of SEQ ID NOs: 17 to 23.

The invention also provides a vector comprising the polynucleotide.

Vectors of the invention include, but are not limited to, plasmid vectors, cosmid vectors, bacteriophage vectors, viral vectors, and the like. The vector of the present invention may be a conventional cloning vector or an expression vector, and the expression vector may comprise a signal sequence or a leader sequence for membrane targeting or secretion in addition to expression control sequences such as a promoter, an operator, an initiation codon, a termination codon, polyadenylation signal and an enhancer (a promoter), and may be variously prepared according to the purpose. The polynucleotide sequence according to the present invention may be operably linked to an expression control sequence, and the operably linked gene sequence and the expression control sequences may be comprised in one expression vector containing a selection marker and a replication origin. “Operably linked” refers to the linkage of an appropriate molecule to an expression control sequence in a manner that enables gene expression, wherein one nucleic acid fragment is linked to another nucleic acid fragment such that its function or expression is affected by the other. “Expression control sequence” means a DNA sequence that regulates the expression of a polynucleotide sequence operably linked in a particular host cell. Such regulatory sequences include a promoter to effect transcription, any operator sequence to control transcription, a sequence encoding a suitable mRNA ribosome binding site, and a sequence that controls the termination of transcription and translation. The vector also includes a selection marker for selecting a host cell containing the vector, and includes a origin of replication if it is a replicable vector.

The invention also provides a transformant transformed with the vector.

Transformation with the vectors can be carried out by transformation techniques known to those skilled in the art. Preferably microprojectile bombardment, electroporation, calcium phosphate (CaPO₄) precipitation, calcium chloride (CaCl₂) precipitation, PEG-mediated fusion, microinjection and liposome-mediated methods can be used.

The term “transformant” may be used interchangeably with “host cell” and refers to a prokaryotic or eukaryotic cell comprising heterologous DNA introduced into the cell by any means (e.g., electroporation, calcium phosphatase precipitation, microinjection, transformation, viral infection, etc.).

In the present invention, the transformant can be used as a host cell for all kinds of single cell organisms commonly used in the cloning field, for example, prokaryotic cells such as various bacteria (e.g., genus Clostridia, E. coli, etc.), lower eukaryotic cells such as yeast and cells derived from higher eukaryotic organisms including insect cells, plant cells, mammals, and the like, but not limited thereto. Since the expression level and modification of the protein appear differently depending on the host cell, a person skilled in the art can select and use the most suitable host cell for the intended purpose.

The present invention also provides a pharmaceutical composition comprising the fusion protein as an active ingredient.

According to one embodiment of the present invention, it has been found that the fusion protein exhibits significantly improved TLR5 pathway activating ability compared to wild type flagellin. Thus, the fusion protein of the present invention may exhibit preventive, ameliorated or therapeutic effects on diseases, syndromes, etc. known to be preventable, ameliorated or treatable through activation of the TLR5 pathway.

Diseases, syndromes known to be preventable, ameliorated or treatable through activation of the TLR5 pathway may be damage by radiation exposure; reperfusion injury; inflammatory bowel disease; autoimmune disease; viral infection; aging; immune function decline; or cancer.

Thus, the pharmaceutical composition of the present invention can be characterized as a pharmaceutical composition for preventing or treating damage caused by radiation exposure; for prevention or treating reperfusion injury; for the prevention or treatment of inflammatory bowel disease; for the prevention or treatment of autoimmune diseases; for preventing or treating viral infections; for prevention or treating aging; for enhancing immune function; or for the treatment of cancer.

In particular, the fusion protein of the present invention may be understood to exert a preventive, ameliorative or therapeutic effect on diseases which will be found in the future to be able to be prevented, ameliorated or treated through TLR5 pathway activation, and thus the disease to be treated of the pharmaceutical compositions of the invention is not particularly limited in scope.

The association of the activation of the TLR5 pathway with the treatment of damage by radiation exposure may refer to KR20067010934A and the like, the association of activation of TLR5 pathways with treatment of tissue damage due to reperfusion may refer to U.S. Pat. No. 8,324,163 and the like, the association of activating TLR5 pathway with treatment for inflammatory bowel disease may refer to U.S. Pat. No. 7,361,733 and the like, the association activating TLR5 pathway with the therapy for autoimmune diseases may refer to EP03010523B1 and the like, the associated with activation TLR 5 pathway with therapy of viral infections may refer to U.S. Pat. No. 9,872,895 and the like, the relationship between activation of the TLR5 pathway and diseases caused by aging may refer to KR20150049811A and the like, the relationship between the activation of the TLR5 pathway and the enhancement of immunity may refer to WO17031280A1 and the like, the relationship between activation of the TLR5 pathway and cancer treatment may refer to KR20177005615A and the like.

In the present invention, the damage by radiation exposure may be a gastrointestinal syndrome or a hematopoietic syndrome due to radiation exposure.

In the present invention, the disease caused by aging may be hair loss due to aging, cataract, hair loss, colitis, osteoporosis or osteomalacia.

In the present invention, the cancer may be breast cancer, lung cancer, colon cancer, kidney cancer, liver cancer, ovarian cancer, prostate cancer, testicular cancer, genitourinary duct cancer, lymphatic system cancer, rectal cancer, pancreatic cancer, esophageal cancer, stomach cancer, cervical cancer, thyroid cancer, skin cancer, leukemia, acute lymphocytic leukemias, acute lymphoblastic leukemia, B-cell lymphoma, T cell lymphoma, Hodgkin lymphoma, non-Hodgkin lymphoma, hairy cell lymphomas, histiocytic lymphoma and Burkitt lymphoma, acute and chronic myelogenous leukemia, myelodysplastic syndrome, myeloid leukemia, promyelocytic leukemia, astrocytoma, neuroblastoma, glioma, schwannoma, fibrosarcoma, rhabdomyosarcoma, osteosarcoma, xeroderma pigmentosum, keratoctanthoma, seminoma, thyroid follicular cancer, teratocarcinoma, or cancer of the gastrointestinal tract.

The pharmaceutical compositions of the present invention may be formulated with a pharmaceutically acceptable carrier in addition to the fusion protein in a manner known in the art according to the route of administration. “Pharmaceutically acceptable” refers to a non-toxic material that is physiologically acceptable and that does not interfere with the action of the active ingredient and does not normally produce an allergic or similar reaction, such as gastrointestinal disorders, dizziness, when administered to a human. Such carriers include all kinds of solvents, dispersion media, oil-in-water or water-oil emulsions, aqueous compositions, liposomes, microbeads and microsomes.

Routes of administration may be orally or parenterally. Parenteral methods of administration include, but are not limited to, intravenous, intramuscular, intraarterial, intramedullary, intrathecal, intracardiac, transdermal, subcutaneous, intraperitoneal, intranasal, enteral, topical, sublingual, or rectal administration.

In the case of oral administration of the pharmaceutical composition of the present invention, it can be formulated into the form of powders, granules, tablets, pills, dragees, capsules, liquids, gels, syrups, suspensions, wafers and the like according to methods known in the art together with a suitable oral carrier. Examples of suitable carriers may include sugars, including lactose, dextrose, sucrose, sorbitol, mannitol, xylitol, erythritol and maltitol and the like, and starches, including corn starch, wheat starch, rice starch and potato starch and the like, celluloses, including cellulose, methyl cellulose, sodium carboxymethylcellulose and hydroxypropylmethylcellulose and the like, and fillers, such as gelatin, polyvinylpyrrolidone, and the like. Also, if desired, cross-linked polyvinylpyrrolidone, agar, alginic acid or sodium alginate and the like may be added as a disintegrant. Furthermore, the pharmaceutical composition may further comprise anti-agglomerants, lubricants, wetting agents, flavoring agents, emulsifying agents, preservatives and the like.

In addition, for parenteral administration, the pharmaceutical composition of the present invention can be formulated in the form of an injection, a transdermal administration and a nasal inhalation, together with a suitable parenteral carrier, according to a method known in the art. In the case of the injection, it must be sterilized and protected from contamination of microorganisms such as bacteria and fungi. Examples of suitable carriers for injection can be solvents or dispersion media including, but not limited to, water, ethanol, polyols (such as glycerol, propylene glycol, and liquid polyethylene glycol and the like), mixtures thereof, and/or vegetable oils. More preferably, as suitable carriers, Hanks' solution, Ringer's solution, phosphate buffered saline (PBS) or isotonic solutions such as sterile water for injection, 10% ethanol, 40% propylene glycol and 5% dextrose, and the like can be used. To protect the injection from microbial contamination, various antibacterial and antifungal agents, such as paraben, chlorobutanol, phenol, sorbic acid, thimerosal and the like, can be further included. In addition, the injection may in most cases further comprise isotonic agents such as sugars or sodium chloride.

In the case of transdermal administration, forms such as ointments, creams, lotions, gels, external solutions, pasta preparations, liniments, aerosols and the like are included. In the above, “transdermal administration” means that a pharmaceutical composition is topically administered to the skin so that an effective amount of the active ingredient contained in the pharmaceutical composition is delivered into the skin. For example, the pharmaceutical composition of the present invention may be administered by a method in which it is prepared in an injectable formulation and the skin is pricked or applied directly to the skin with a 30-gauge fine injection needle. These formulations are described in a formulation generally known in pharmaceutical chemistry.

In the case of inhalation administration, the compounds used in accordance with the invention can be conveniently delivered in the form of an aerosol spray from pressurized packs or a nebulizer, with the use of a suitable propellant, e.g., dichlorofluoromethane, trichlorofluoromethane, dichlorotetrafluoroethane, carbon dioxide or other suitable gas. In the case of a pressurized aerosol, the dosage unit may be determined by providing a valve to deliver a metered amount. For example, gelatin capsules and cartridges for use in an inhaler or insufflator may be formulated to contain a powder mix of the compound and a suitable powder base such as lactose or starch.

As other pharmaceutically acceptable carriers, those known in the art can be referred to.

In addition, the pharmaceutical compositions according to the present invention may further comprise one or more buffers (such as saline or PBS), carbohydrates (such as glucose, mannose, sucrose, or dextran), antioxidants, bacteriostats, chelating agents (such as EDTA or glutathione), adjuvants (such as aluminum hydroxide), suspending agents, thickening agents, and/or preservatives.

The pharmaceutical compositions of the invention may also be formulated using methods known in the art to provide quick, sustained or delayed release of the active ingredient after administration to a mammal.

In addition, the pharmaceutical composition of the present invention can be administered in combination with known substances having the effect of preventing or treating the respective diseases listed above.

The present invention also provides a vaccine adjuvant comprising the fusion protein as an active ingredient.

One of the most important requirements of vaccine adjuvants is to have immunomodulatory functions such as regulation of costimulatory molecule expression on the surface of antigen presenting cells and regulation of cytokine secretion due to induction of antigen-specific T cells.

By the way, PRRs such as TLR5 are distributed in the cell surface or cytoplasm of host cells, and induce ‘innate immune response’ by stimulation of various PAMPs, and further regulate ‘adaptive immune response.’ Thus, TLR5 agonists may be suitable targets for the development of various ‘immunomodulators’, particularly ‘vaccine adjuvants’.

Thus, the fusion proteins of the present invention that have the ability to activate the TLR5 pathway can activate TLR5 pathways to enhance innate immune responses and acquired immune responses, so that the host's immunity to the co-administered antigen can be significantly improved.

The vaccine adjuvants of the present invention may be prepared by conventional methods well known in the art, and may optionally further comprise various additives that may be used in preparing vaccines in the art.

The present invention also provides a use of the fusion protein for the preparation of a pharmaceutical agent for the treatment of injury by radiation exposure; the treatment of reperfusion injury; the treatment of inflammatory bowel disease; the treatment of autoimmune disease; the treatment of viral infection; the treatment of metabolic disease; the treatment of aging; the enhancement of immune function; or the treatment of cancer.

In addition, the present invention provides a method for treating injury by exposure to radiation; treating reperfusion injury; treating inflammatory bowel disease; treating autoimmune disease, treating viral infection; treating metabolic disease; treating aging; enhancing immune function; or treating cancer, comprising administering to a subject in need thereof an effective amount of a composition comprising the fusion protein as an active ingredient.

The ‘effective amount’ of the present invention refers to an amount that, when administered to a subject, exhibits the effect of improving, treating, preventing, detecting, diagnosing, or inhibiting or reducing the damage caused by radiation exposure; reperfusion injury; inflammatory bowel disease; autoimmune diseases; viral infection; metabolic diseases; aging; boosting immune function; or cancer. The ‘subject’ may be an animal, preferably a mammal, in particular an animal including a human, and animal derived cells, tissues, organs, etc. The subject may be a patient in need of the effect.

The ‘treatment’ of the present invention refers broadly to ameliorating injury by radiation exposure; reperfusion injury; inflammatory bowel disease; autoimmune disease; viral infection; metabolic disease; aging; immune function enhancement; or cancer, or symptoms of the disease, which may include, but are not limited to, curing, substantially preventing, or ameliorating the condition of, and which includes ameliorating, curing or preventing one or most symptoms resulting from the disease.

As used herein, the term ‘comprising’ is used in the same sense as ‘including’ or ‘characterized by’, and in the composition or method according to the present invention, additional components or steps of the method not specifically mentioned are not excluded. In addition, the term ‘consisting of’ means excluding additional elements, steps, or ingredients that are not separately described. The term ‘essentially consisting of’ means that, in the scope of the composition or method, it may include substances or steps that do not substantially affect the basic properties thereof in addition to the substances or steps described.

### Advantageous Effect

The fusion protein provided by the present invention has significantly superior toll-like receptor 5 (TLR5) pathway activation ability compared to a wild-type flagellin, a fragment thereof or a variant thereof, and it can be very usefully utilized in the development of therapeutic agents and/or vaccine adjuvants for diseases that can be prevented, improved, or treated through activation of the TLR5 pathway.

### BRIEF DESCRIPTION OF THE DRAWINGS

FIG. 1 is a schematic diagram simply showing the preparation of Fc fusion flagellin of various microorganisms-derived flagellin and human IgG4-Fc (hIgG4) provided in the present invention.

FIG. 2 is the result of confirming cloning by culturing colonies of each strain cutting with enzymes, and checking the size of flagellin and vector of each strain to confirm the fusion of the prepared microorganism-derived Fc-hIgG4-flagellin (BS, BsFlagellin; EC, EcFlagellin; PA, PaFlagellin; SD, SdFlagellin; SH, ShFlagellin).

FIG. 3 is a result showing that each Fc-hIgG4-flagellin fusion protein forms a dimer.

FIG. 4 is a result showing the comparison of TLR5-dependent NF-κB activity by Fc-hIgG4-flagellin fusion proteins derived from each microorganism.

FIG. 5 is a schematic diagram of the production of Fc-mIgG1-Bsflagellin prepared for animal experiments of Bsflagelllin, which induces the highest TLR5 activity. A fusion protein was constructed using two linkers of different lengths.

FIG. 6 is a result confirming the cloning of Fc-mIgG1-Bsflagellin prepared using two linkers of different lengths.

FIG. 7 is a result of confirming the expression of the fusion protein of the present invention through Western blot (#1: Fc-mIgG1-linker-bsflagellin, #2: Fc-mIgG1-linker*3-bsflagellin).

FIG. 8 is a result of confirming through Western blot whether the fusion protein of the present invention forms a dimer.

FIG. 9 is a result of evaluating (FLA-ST) NF-κB activation ability of an Fc-fused bsflagellin (mIgG1-Fc2-Bsflagellin) and purified flagellin protein (FLA-BS) of Bacillus subtilis, and purified flagellin protein of Salmonella typhimurium.

### MODE FOR CARRYING OUT INVENTION

Hereinafter, the present invention will be described in detail.

However, the following examples are only illustrative of the present invention, and the content of the present invention is not limited to the following examples.

### Example 1: Preparation of Fc-Flagellin Fusion Protein (Cloning)

(1) Experimental Method

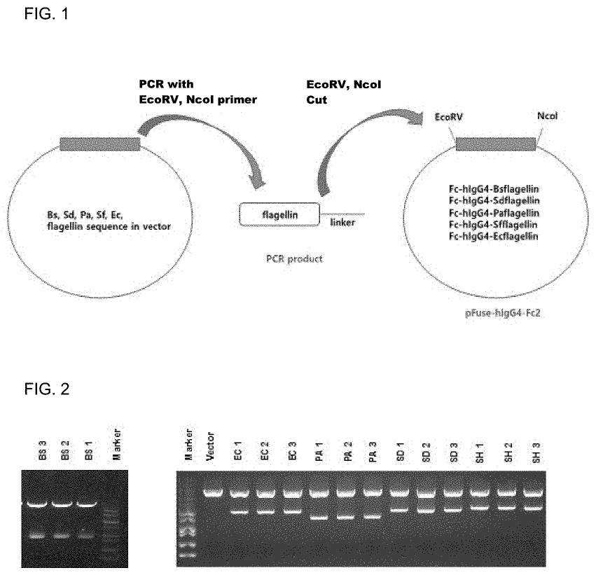

It was attempted to produce an Fc-flagellin fusion protein in which flagellin derived from various microorganisms and the Fc region of an antibody were fused (FIG. 1).

As shown in FIG. 1, cloning was performed according to the following method in order to construct plasmids each expressing a form in which flagellin derived from Bacillus subtilis (Bs), Salmonella dublin (Sd), Pseudomonas aeruginosa (Pa), Shigella flexneri (Sf) and Escherichia coli (Ec) was bound to human IgG4-Fc (hIgG4).

1) PCR was performed for all flagellins using EcoRV and NcoI restriction enzyme site primers (Table 2) according to the conditions shown in Table 1 below:

TABLE 1

| | | EcoRV- | EcoRV- | EcoRV- | EcoRV- | EcoRV- |
|---|---|---|---|---|---|---|
| | Negative | Bsflagelli | Ecflagelli | Sdflagelli | Sfflagelli | Paflagelli |
| | Control | n-Ncol | n-Ncol | n-Ncol | n-Ncol | n-Ncol |
| 10X | 5 uL | 5 uL | 5 uL | 5 uL | 5 uL | 5 uL |
| Buffer | | | | | | |
| dNTP | 1 uL | 1 uL | 1 uL | 1 uL | 1 uL | 1 uL |
| Forward | 2 uL | 2 uL | 2 uL | 2 uL | 2 uL | 2 uL |
| Primer | | | | | | |
| Reverse | 2 uL | 2 uL | 2 uL | 2 uL | 2 uL | 2 uL |
| Primer | | | | | | |
| Template | 1 uL | 1 uL | 1 uL | 1 uL | 1 uL | 1 uL |
| PCR | 0 uL | 1 uL | 1 uL | 1 uL | 1 uL | 1 uL |
| Enzyme | | | | | | |
| DW | Up to 50 uL | Up to 50 uL | Up to 50 uL | Up to 50 uL | Up to 50 uL | Up to 50 uL |

PCR Cycle
1. 95° C. for 2 min
2. 95° C. for 20 secs
3. 65° C. for 40 secs
4. 72° C. for 1 min à go to step 2 × 29
5. 72° C. for 5 min

TABLE 2

| Division | Primer sequence |
|---|---|
| Ecflagellin-hIgG4-Fc2- | 5′ CCGGATATCGAT GGC ACA AGT CAT TAA 3′ |
| 5′ EcoR V -G | TAC CAA CAG CCT CTC G SEQ ID NO: 24) |
| Ecflagellin-(GGGGS)- | 5′ GACCATGGC (AGA CCC TCC GCC ACC) 3′ |
| hIgG1-Fc2-3′ Nco I -C | CTG CAG CAG AGA CAG AAC CTG |
| | CTG (SEQ ID NO: 25) |
| Paflagellin-hIgG4-Fc2- | 5′ CCGGATATCGAT GGC CTT GAC CGT CAA 3′ |
| 5′ EcoR V -G | CAC CAA CAT CGC TTC G (SEQ ID NO: 26) |
| Paflagellin-(GGGGS)- | 5′ GACCATGGC (AGA CCC TCC GCC ACC) 3′ |
| hIgG1-Fc2-3′ Nco I -C | GCG CAG CAG GCT CAG AAC CGA CTG |
| | CGG (SEQ ID NO: 27) |
| Sdflagellin-hIgG4-Fc2- | 5′ CCGGATATCGAT GGC ACA AGT CAT TAA 3′ |
| 5′ EcoR V -G | TAC AAA CAG C (SEQ ID NO: 28) |
| Sdflagellin-(GGGGS)- | 5′ GACCATGGC (AGA CCC TCC GCC ACC) 3′ |
| hIgG1-Fc2-3′ Nco I -C | ACG CAG TAA AGA GAG GAC GTT |
| | TTG (SEQ ID NO: 29) |
| Sfflagellin-hIgG4-Fc2- | 5′ CCGGATATCGAT GGC ACA AGT CAT TAA 3′ |
| 5′ EcoR V -G | TAC CAA CAG C (SEQ ID NO: 30) |
| Sfflagellin-(GGGGS)- | 5′ GACCATGGC (AGA CCC TCC GCC ACC) 3′ |
| hIgG1-Fc2-3′ Nco I -C | ACC CTG CAG CAG AGA CAG AAC |
| | CTG (SEQ ID NO: 31) |
| Bsflagellin-hIgG4-Fc2- | 5′ C CGGATATCG ATG AGA ATT AAC CAC 3′ |
| 5′ EcoR V -G | AAT ATT GCA GCA CTT AAC (SEQ ID NO: 32) |
| Bsflagellin-(GGGGS)- | 5′ GACCATGGC (AGA CCC TCC GCC ACC) 3′ |
| hIgG1-Fc2-3′ Nco I -C | ACG TAA TAA TTG AAG TAC GTT TTG AGG |
| | CTG (SEQ ID NO: 33) |

✓ Bold type G and C are the nucleotide sequences inserted to match the Open Reading Frame

2) After adding 4.4 uL of 1 Ox DNA dye to the PGR product, gel electrophoresis was performed, the size was checked, and the flagellin band was extracted using MEGAquick-Spin™ plus (cat #17290).

3) Purified DNA was digested with EcoRV and NGoI according to the conditions in Table 3 below:

TABLE 3

| | | EcoRV- | EcoRV- | EcoRV- | EcoRV- | EcoRV- |
|---|---|---|---|---|---|---|
| | pFUSE- | Bs- | Es- | Sd- | Sf- | Pa- |
| | hIgG4- | flagelli | flagelli | flagelli | flagelli | flagelli |
| | Fc2 | n-Ncol | n-Ncol | n-Ncol | n-Ncol | n-Ncol |
| 10X K | 4 uL | 4 uL | 4 uL | 4 uL | 4 uL | 4 uL |
| DNA | 1 ug | 1 ug | 1 ug | 1 ug | 1 ug | 1 ug |
| EcoRV | 1 uL | 1 uL | 1 uL | 1 uL | 1 uL | 1 uL |
| Ncol | 1 uL | 1 uL | 1 uL | 1 uL | 1 uL | 1 uL |
| BSA 0.1% | 2 uL | 2 uL | 2 uL | 2 uL | 2 uL | 2 uL |
| DW | Up to | Up to | Up to | Up to | Up to | Up to |
| | 40 uL | 40 uL | 40 uL | 40 uL | 40 uL | 40 uL |

For 20 hrs at 37° C.

4) 1 ul of Gaff Alkaline Phosphatase was added to the vector cut sample and reacted at 37° C. for 2 hours.

5) After gel electrophoresis by adding 4.4 uL of 10×DNA dye, each band was extracted using MEGAquick-Spin™ plus (cat #17290).

6) Molecular ratio Vector: Insert=1:3, and ligation was performed according to the conditions in Table 4 below.

TABLE 4

| | | | | | | Nagative |
|---|---|---|---|---|---|---|
| | V + Bs | V + Ec | V + Sd | V + Sf | V + Pa | control |
| 10X B | 1 uL | 1 uL | 1 uL | 1 uL | 1 uL | 1 uL |
| Vector | 1 uL | 1 uL | 1 uL | 1 uL | 1 uL | 1 uL |
| Ligase | 0.5 uL | 0.5 uL | 0.5 uL | 0.5 uL | 0.5 uL | 0.5 uL |
| Insert | 1.5 uL | 3.15 uL | 3.51 uL | 2.92 uL | 3.80 uL | 0 uL |
| DW | Up to 10 uL | Up to 10 uL | Up to 10 uL | Up to 10 uL | Up to 10 uL | Up to 10 uL |

For 16 hrs at 16° C.
*Vector Conc.: 4.5/4.1 kb
Bs: 2.5/845 bp
Ec: 1.5/1.5 kb
Sd: 2.5/1.5 kb
Sf: 2.4/1.6 kb
Pa: 2.5/1.2 kb

7) Transformation was carried out in Zeocin(+) LB plate and cultured for 18 hours.

8) Mini Prep was performed and enzymatic digestion was performed using EcoRV and NcoI.

(2) Experiment Results

First, as a result of gel electrophoresis by picking three colonies each, as shown in FIG. 2, all five types of Bsflagellin, Ecflagellin, Paflagellin, Sdflagellin, and Shflagellin were confirmed to have the same vector size, and it was confirmed that the sequence was 100% identical without mutation through sequencing.

### Example 2: Preparation of Fc-higG4-Flagellin Fusion Protein (Separation and Purification)

(1) Experimental Method
5 mL of Protein A Resin [Repligen, #10-2500-01] is washed three times in total with 20 mL of Binding Buffer (pH 5.0; contains 0.02% sodium azide) [Thermo Fisher Scientific, 21019].

1) Resin is allowed to naturally settle for 5 minutes each time upon washing, and then the supernatant is removed.
The washed Resin is slowly poured into Disposable Columns [Thermo Fisher Scientific, 29920]. The Column and Resin are brought to the same state by pouring a Binding Buffer into the prepared Column.
The cell culture prepared at room temperature is then slowly poured into the Column. The solution is then slowly flowed out of the Column. Once all of the cell culture is removed, the Column is washed with Binding Buffer.
After the neutralization solution (1 M Tris, pH 9.2) was pre-soaked in a microfuge tube prior to elution, Column was put into the prepared microfuge tube and Elution Buffer (pH 2.8; amine-based) [Thermo Fisher Scientific, 21004] was put in good mixing with Resin. After waiting until all of the Resin sinks, the solution is allowed to flow slowly.
Spin Desalting Column [Thermo Fisher Scientific, 89891] is used to change the buffer solution of the isolated protein to Phosphate Buffered Saline (PBS).

1) Equilibrate 5 ml PBS in Column for a total of 5 times, then slowly pour the extracted protein solution into Column and run a portion of the initial solution. The PBS solution is again added to the Column and the solution is slowly added to a new tube.
Endotoxin present in the protein solution is removed using Endotoxin Removal Spin Column [Thermo Fisher Scientific, 88273].

1) The Spin Column is placed in a tube and centrifuged at a speed of 500 g for 1 minute to evacuate the solution contained therein, followed by 2M Sodium Chloride (NaCl) and slowly shaking the Column until the Resin was suspended in the solution. Column is placed in a tube and centrifuged for 1 minute at a speed of 500 g to evacuate the solution contained therein.

2) Adding pure water free of endotoxin to the Resin, gently shaking the Column until the solution is suspended, then placing the Column in a tube and centrifuging for 1 minute at a speed of 500 g to evacuate the solution contained therein.

3) Thereafter, Buffer (25 mM Sodium Phosphate, pH 7.0) without endotoxin was added and Column was gently shaken until the Resin was suspended in the solution. Column is placed to the tube and centrifuged at 500 g speed for 1 minute to evacuate the solution contained therein. This process is repeated twice.

4) The extracted protein solution is slowly poured and the Column is gently shaken until the Resin is suspended in the solution, followed by incubation at 4° C. for 1 hour with gentle ramp-down.

5) The Column is placed in a tube and centrifuged at a speed of 500 g for 1 minute to obtain the solution contained in the tube.
The degree of endotoxin of the purified protein solution is measured using the LAL Chromogenic Endotoxin Quantitation Kit [Thermo Fisher Scientific, 88282].

1) To measure the standard curve in a 96-well plate at 37° C., 50 μL each of pure water without endotoxin (0 EU/mL) and standard protein solution (0.1, 0.25, 0.5, 1 EU/ml) are added to the wells.

2) 50 μL of the purified protein solution is added to two wells per sample.

3) 50 μL of Limulus Amebocyte Lysate (LAL) reagent is added to each well, and then the plate is gently shaken and mixed and incubated at 37° C. for 10 minutes. After 10 minutes, 100 μL of the Chromogenic Substrate reagent was added to each well, and the plate was gently shaken to mix and incubated at 37° C. for 6 minutes. After 6 minutes, 100 μL of Stop Reagent (25% acetic acid) was added to each well and the plate was gently mixed at room temperature.

4) The optical density (OD) value is measured directly on a micro-multireader machine with a wavelength of 410 nm.

5) After the standard curve is drawn with the OD value obtained in the standard protein solution, the fitting function in exponential form is identified, and then the OD values obtained from the purified protein solution are fitted to the fitting function to determine the endotoxin concentration.
The concentration of the final harvested purified protein solution is measured using the BCA Protein Assay Kit [Thermo Fisher Scientific, 23227].

1) To a 96-well plate, a purified protein solution, PBS (0 ug/mL), and a standard protein solution were serially diluted in PBS to prepare standard protein solutions (25, 125, 250, 500, 750, 1000, 1500, and 2000 ug/mL) each at a concentration of 10 μL per well.

2) Solution A and Solution B enclosed in Kit were mixed at a ratio of 50:1, 200 μL each was added to each well, and the mixture was incubated for 30 minutes in a 37° C. incubator without light irradiation.

3) The OD value is measured immediately after 30 minutes in a micro multireader with a wavelength of 562 nm.

4) The protein concentration is determined by determining the fitting function in exponential form after a standard curve is drawn with the OD value obtained in the standard protein solution and then substituting the OD value obtained in the purified protein solution into a fitting function.

(2) Experimental Results

The concentration of each protein after separation and purification according to the above experimental method is shown in Table 5.

TABLE 5

| | Plasmid | Trans- | | | Endotoxin |
|---|---|---|---|---|---|
| | DNA | fection | Quantification | | level |
| | prep | (100 mL) | mg | mg/L | (EU/mL) |
| Fc-hlgG4-Ecflagellin | Done | Done | 0.25 | 2.5 | 12.3 |
| Fc-hlgG4-Sdflagellin | Done | Done | 0.41 | 4.1 | 6.8 |
| Fc-hlgG4-Sfflagellin | Done | Done | 0.08 | 0.8 | 0.1 |
| Fc-hlgG4-Bsflagellin | Done | Done | 2.15 | 21.5 | 7.4 |
| Fc-hlgG4-Paflagellin | Done | Done | 0.96 | 9.6 | 11.0 |

### Example 3: Confirmation of Dimerization of Fusion Proteins

An attempt was made to determine whether the Fc-flagellin fusion protein of various strains in which the expression of the protein was confirmed forms a dimer.

(1) Experimental Method
Cell line used: HEK293T
24 well plates are seeded to a cell confluence of 70% on the day of transfection.
On the day of the experiment, JetPrime transfection reagent was mixed with 500 ng of each DNA (EV, #1, #2, TdTmt) in an amount of 1 ul per well, followed by transfection according to the protocol (for transfection, 10% FBS DMEM was used. Transfection volume is 500 ul).
24 hours after transfection, it is switched to 0.5% FBS media.
harvest the media 400 ul after 48 hours.
   Media is centrifuged at 12,300 g for 1 minute to remove debris and the supernatant is used as a sample.
50 ul of two kinds of 5× sample buffer are added by dividing each sample by 200 ul.

1) Reducing 5× sample buffer: a commonly used Laemmli sample buffer composition

2) Non-Reducing 5× sample buffer: no β-mercaptoethanol was added in the Laemmli sample buffer composition.
Heat reducing samples at 99° C. heat block for 5 minutes.
Non-reducing samples omit the heating process
Separate by size of protein by SDS-PAGE.
Check the size by Coomasie staining.

(2) Experimental Results

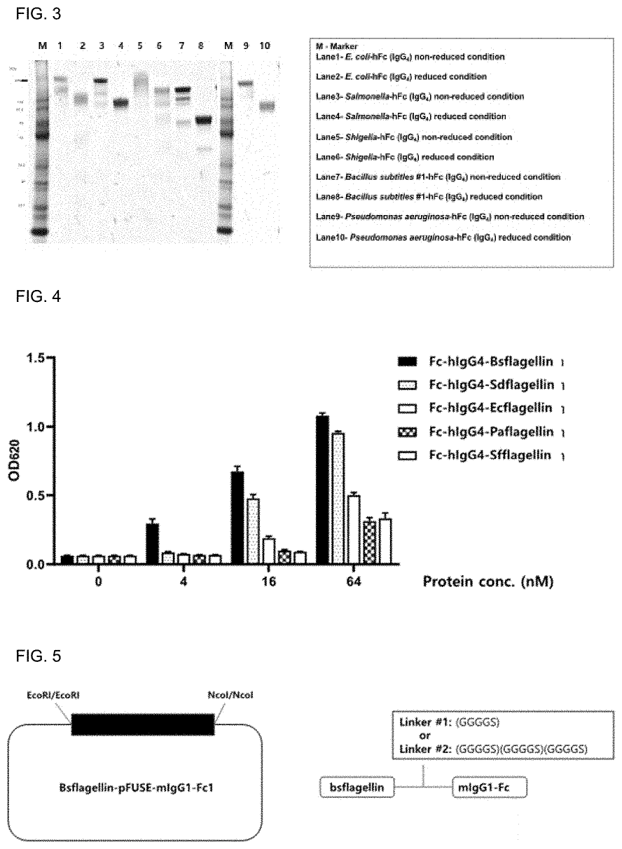

The results are shown in FIG. 3. Under Non-reducing conditions, a dimer formation signal corresponding to approximately twice the size from which the signal appeared at reducing conditions was observed for each Fc-flagellin fusion protein. This confirmed that the Fc-flagellin fusion protein forms a dimer.

### Example 4: Evaluation of TLR5 Pathway Activation Ability of Fc-Flagellin Fusion Protein

In order to confirm whether the prepared Fc-flagellin fusion protein exhibits activity as a TLR5 agonist, intracellular NF-κB activity was evaluated. As the cells, HEK-Blue hTLR5 (Invivogen, Cat No. hkb-htrl5), which is a cell line model constructed for selecting an agent that induces TLR5 activity to HEK293T, was used, and TLR5 activities by Fc-flagellin prepared by overexpression of hTL R5 and pNF-κB can be confirmed.

(1) Experimental Method

HEK-Blu hTLR5 cells are maintained in culture medium containing Zeocin (100 ug/mL) (Invivogne, Cat No. ant-zn-1), blasticidin (15 ug or mL) (Invivogen, Ct No. at-bl-1), and normocin (100 ug/mL (invivoogen, C at No. anti-nr-1).
The method for measuring NF-kB activity is as follows:

\textcircled{1} The cultured HEK-Blue hTLR5 cells are detached, centrifuged, the supernatant is removed, and the cell pellet is mixed well with 1×PBS.

\textcircled{2} Prior to seeding the cells, pre-prepared protein samples (20 ul) at concentrations of 4 nM, 16 nM, and 64 nM, respectively, are added to 96-well plates in advance.

\textcircled{3} The experiment was conducted under Tri-plates conditions.

\textcircled{4} The control group is a group added with 1×PBS (20 ul).

\textcircled{5} Calculated HEK-Blue hTLR5 cells were mixed well with HEK-BLUE Dection medium (Invivogen, Cat No. hb-det2) and seeded in 96-well plates at approximately 2.5×10⁴/96-well with equal cell numbers in 180 ul volume.

\textcircled{6} Cells added with protein are reacted in a 37° C. incubator for about 16 hours.

\textcircled{7} After about 16 hours, the NF-kB activity is compared and calculated by measuring the optical density (OD) values at 620 nm wavelength in a micro multireader.

(2) Experimental Results

The results are shown in FIG. 4.

As shown in FIG. 4, each of the Fc-hIgG4-flagellin fusion proteins was found to increase TLR5-dependent NF-κB activity in a concentration-dependent manner, and in particular, it was confirmed that the activity of the Fc-hIgG4-bsflagellin fusion protein was the most excellent at all concentrations. It was found that other Fc-hIgG4-flagellin fusion proteins at low concentrations of 4 nM do not induce the activity of TLR5, whereas only Fc-HIgG4-bs flagellin is capable of inducing significant TLR5 activity.

### Example 5: Preparation of Fc-mIgG1-Bsflagellin Fusion Protein for Animal Experiments

In order to validate the Fc-hIgG4-Bsflagellin identified as showing the most excellent activity in Examples 1 to 4 above in a mouse animal model, a vector comprising a polynucleotide encoding a protein fused with an Fc region derived from mouse IgG1 was prepared as follows (FIG. 5). In order to compare whether the linker size affects the function of Fc fusion flagellin, one linker GGGGS (SEQ ID NO: 8) and three linkers GGGS (Seq. ID NO: 9) were used, respectively.

A vector comprising a polynucleotide encoding a protein fused with a Bsflagelliin, a linker and an Fc region derived from mouse IgG1 was prepared as follows (FIG. 5).

Specifically, PCR was performed in a pET49b-bsflagellin vector (Scientific Reports, 7, 40878 (2017)) in which the B. subtilis flagellin (bs flagelin) gene was inserted using the forward primer (5′-CCG GAATTCATGAGAATTAACCACAATATTGCAGCACTTAAC-3) (SEQ ID NO: 34) and the reverse primers (5′-GACCATGGTAGACCCTCCGCCACCACGTAATAATTGAAGTACGTTTTTGAGGCTG-3′ (SEQ NO: 35) and 5′-GACCATGGTAGACCCTCCACCACCACGACCTCCGCCACCACGTAATAATTGAGTAC GTTTGAGGCTG-3′-(SEQ ID No: 36)). PCR is performed using pfu turbo DNA polymerase (Invitrogen, 2.5 unit/ul, #600252) at 92° C. for 30 seconds, at 92° C. for 30 seconds, at 56° C. for 30 seconds, at 68° C. for 1 minute as 1 cycle. After 30 cycles, the reaction was terminated by reacting at 68° C. for 5 minutes.

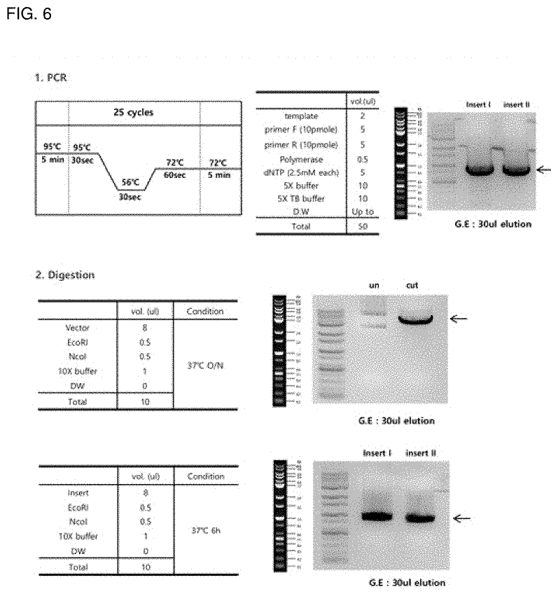

The PCR product thus obtained was treated with EcoR1/NcoI and then ligated into the pFUSE-mIgG1-Fc1 vector treated with the EcoR1/Nco I enzyme using T4 DNA ligase (Takara, #2011A) to confirm the expression vector of “pFUSE-mIgG1-Fc1-bs flagellin” by sequencing (FIG. 6).

The expression of the Fc-Bsflagellin fusion protein was confirmed after transfection of cells with the vector prepared above.

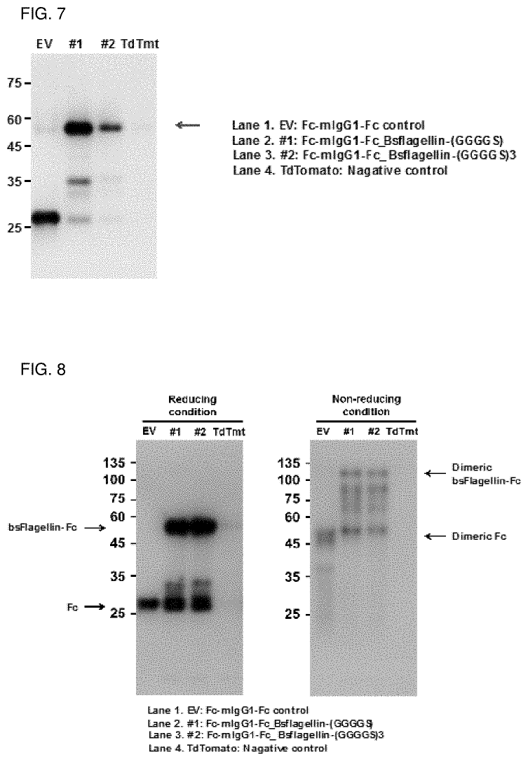

The results are shown in FIG. 7.

As can be seen from FIG. 7, unconjugated Fc protein (26 kDa) was identified in EVs, and Bsflagellin conjugated Fc proteins were identified in #1 (Fc-bs flagellin, i.e., Fc-linker-bs flagellin fusion protein) and #2 (Fc-bsflagellin3, i.e., Fc-linker 3 repeat-bs flagellin fusion protein), respectively, at a size of 55 kDa. No signal was detected in TdTmt lane used for Negative control. The Fc-bsflagellin fusion protein is a fusion protein of the bsflagellin-linker (GGGGS (SEQ ID NO: 8))-Fc structure, and the Fc-bsflagellin3 refers to a fusion proteins of a bsflagellin-linker (GGGGS 3 repeats (SEQ ID No: 9))-Fc structure.

### Example 6: Confirmation of Dimerization of Fc-mIgG1-Bsflagellin Fusion Protein

An attempt was made to determine whether the Fc-mIgG1-bsflagellin fusion protein, whose expression was confirmed, forms a dimer.

As shown in FIG. 8, under Non-reducing conditions, a dimer formation signal corresponding to about twice the size from which the signal emerges at reducing conditions was observed. This confirmed that the Fc-bsflagellin fusion protein forms a dimer.

### Example 7: Comparison of the Activities of Fc-Flagellin and Wild-Type Flagellin as TLR5 Agonists

To confirm that the Fc-bsflagellin fusion protein prepared above exhibits activity as a TLR5 agonist, the intracellular NF-κB activity was evaluated in the same manner as in Example 4.

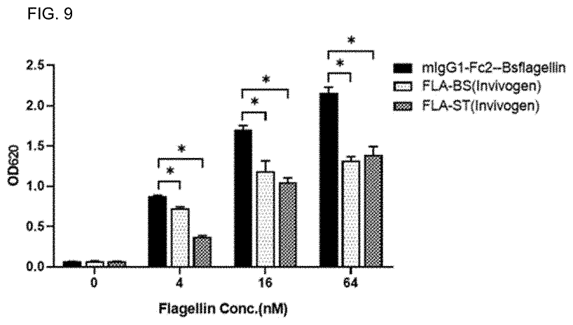

The results are shown in FIG. 9.

The activity of Toll-like receptor (TLR) 5-dependent NF-κB was confirmed with cell culture obtained 48 hours after transfection of Fc-mIgG1-Bsflagellin into HEK293T cells. For comparison, purified flagellin (FLA-BS) from Bacillus subtilis and purified flagellin (FLA-ST) from Salmonella typhimurium, sold at 99% purity by Invivogen, were used. When each test substance was treated, it was confirmed that the TLR5-dependent NF-κB activity by Fc-bsflagellin was significantly higher than that of FLA-BS or FLAs-ST used as a standard protein.

The above results indicate that flagellin fused to the Fc region of an immunoglobulin exhibits significantly improved TLR5 pathway activation ability as compared to wild-type flagellins, fragments of wild type flagelline or variants of wild-type flagellin.

The amino acid sequence of each protein used in the above experiment and the sequence of the polynucleotide encoding it are as follows:

Bsflagellin amino acid sequence: SEQ ID NO: 1

Sdflagellin amino acid sequence: SEQ ID NO: 2

Paflagellin amino acid sequence: SEQ ID NO: 3

Shflagellin amino acid sequence: SEQ ID NO: 4

Ecflagellin amino acid sequence: SEQ ID NO: 5

Human IgG4 Fc amino acid sequence: SEQ ID NO: 6

Mouse IgG1 Fc amino acid sequence: SEQ ID NO: 7

Linker 1 amino acid sequence: SEQ ID NO:8

Linker 2 amino acid sequence: SEQ ID NO: 9

hIgG4-Fc-Bsflagellin fusion protein amino acid sequence: SEQ ID NO: 10

mIgG1-Fc-Bsflagellin fusion protein (Linker 1) amino acid sequence: SEQ ID NO: 11

mIgG1-Fc-Bsflagellin fusion protein (Linker 2) amino acid sequence: SEQ ID NO: 12

hIgG4-Fc-Sdflagellin fusion protein amino acid sequence: SEQ ID NO: 13

hIgG4-Fc-Paflagellin fusion protein amino acid sequence: SEQ ID NO: 14

hIgG4-Fc-Shflagellin fusion protein amino acid sequence: SEQ ID NO: 15

hIgG4-Fc-Ecflagellin fusion protein amino acid sequence: SEQ ID NO: 16

hIgG4-Fc-Bsflagellin polynucleotide sequence: SEQ ID NO: 17

mIgG1-Fc-Bsflagellin (Linker 1) polynucleotide sequence: SEQ ID NO: 18

mIgG1-Fc-Bsflagellin (Linker 2) polynucleotide sequence: SEQ ID NO: 19

hIgG4-Fc-Sdflagellin polynucleotide sequence: SEQ ID NO: 20

hIgG4-Fc-Paflagellin polynucleotide sequence: SEQ ID NO: 21

hIgG4-Fc-Shflagellin polynucleotide sequence: SEQ ID NO:22

hIgG4-Fc-Ecflagellin polynucleotide sequence: SEQ ID NO:23

### INDUSTRIAL APPLICABILITY

The fusion protein provided by the present invention has significantly superior toll-like receptor 5 (TLR5) pathway activation ability compared to wild-type flagellin, a fragment thereof or a mutant thereof, and it can be very usefully utilized in the development of therapeutic agents for diseases that can be prevented, improved, or treated through activation of the TLR5 pathway and/or vaccine adjuvant development, and thus has very high industrial applicability.

1. A fusion protein comprising a flagellin, a fragment thereof, or a variant thereof; and an immunoglobulin Fc region.

2. The fusion protein according to claim 1, wherein the flagellin is flagellin derived from a microorganism selected from the group consisting of the genus Bacillus, Salmonella, Helicobacter, Vibrio, Serratia, Shigella, Treponema, Legionella, Borrelia, Clostridium, Agrobacterium, Bartonella, Proteus, Pseudomonas, Escherichia, Listeria, Yersinia, Campylobacter, Roseburia, and Marinobacter.

3. The fusion protein according to claim 1, wherein the flagellin is flagellin derived from a microorganism selected from the group consisting of Salmonella enteritidis, Salmonella typhimurium, Salmonella Dublin, Salmonella enterica, Helicobacter pylori, Vibrio cholera, Vibrio vulnificus, Vibrio fibrisolvens, Serratia marcesens, Shigella flexneri, Treponema pallidum, Borrelia burgdorferei, Clostridium difficile, Agrobacterium tumefaciens, Bartonella clarridgeiae, Proteus mirabilis, Bacillus subtilis, Bacillus cereus, Bacillus halodurans, Pseudomonas aeruginosa, Escherichia coli, Listeria monocytogenes, Yersinia pestis, Campylobacter spp, Roseburia spp and Marinobacter spp.

4. The fusion protein according to claim 1, wherein the flagellin comprises a conserved sequence recognized by toll-like receptor 5 (TLR5).

5. The fusion protein according to claim 1, wherein the fragment has a hypervariable region removed from wild-type flagellin.

6. The method of claim 1, wherein the fragment comprises at least one selected from the group consisting of C-terminal domain 0, C-terminal domain 1, C-terminal domain 2, N-terminal domain 2, N-terminal domain 1, N-terminal domain 0 of wild type flagellin and a domain exhibiting at least 80% amino acid sequence homology with each of the domains.

7. The fusion protein according to claim 1, wherein the variant shows at least 80% amino acid sequence homology with wild-type flagellin and exhibits Toll-like receptor 5 (TLR5) stimulating activity.

8. The fusion protein according to claim 1, wherein the immunoglobulin Fc region is derived from an Fc of human or animal immunoglobulin IgG, IgM, IgD, IgA or IgE.

9. The fusion protein according to claim 1, wherein the immunoglobulin Fc region is derived from an Fc of human or animal immunoglobulin IgG1, IgG2, IgG3 or IgG4.

10. The fusion protein according to claim 1, wherein the immunoglobulin Fc region comprises at least one selected from the group consisting of CH1, CH2, CH3 and CH4 domains.

11. The fusion protein according to claim 10, wherein the immunoglobulin Fc region further comprises a hinge region.

12. The fusion protein according to claim 1, wherein the N-terminus or C-terminus of the flagellin, the fragment thereof or the variant thereof is bound to the N-terminus or C-terminus of the immunoglobulin Fc region.

13. The fusion protein according to claim 1, wherein the flagellin, the fragment thereof, or the variant thereof; and the immunoglobulin Fc region is linked via a linker.

14. The fusion protein according to claim 1, wherein the flagellin, the fragment thereof, or the variant thereof consists of an amino acid sequence selected from the group consisting of SEQ ID NOs: 1 to 5 or an amino acid sequence exhibiting at least 80% sequence homology thereto.

15. The fusion protein according to claim 1, wherein the immunoglobulin Fc region consists of the amino acid sequence of SEQ ID NO: 6 or 7 or an amino acid sequence exhibiting at least 80% sequence homology thereto.

16. The fusion protein according to claim 13, wherein the linker consists of the amino acid sequence of SEQ ID NO: 8 or SEQ ID NO: 9.

17. The fusion protein according to claim 1, wherein the fusion protein consists of an amino acid sequence selected from the group consisting of SEQ ID NOs: 10 to 16.

18. A polynucleotide encoding the fusion protein of claim 1.

19. (canceled)

20. (canceled)

21. A composition or a vaccine adjuvant comprising the fusion protein of claim 1 as an active ingredient.

22. (canceled)

23. (canceled)

24. (canceled)

25. (canceled)

26. (canceled)

27. (canceled)

28. A method for treating injury by exposure to radiation; treating reperfusion injury; treating inflammatory bowel disease; treating autoimmune disease, treating viral infection; treating metabolic disease; treating aging; enhancing immune function; or treating cancer, comprising administering to a subject in need thereof an effective amount of a composition comprising the fusion protein of claim 1 as an active ingredient.

29. The method of claim 28, wherein the composition exhibits Toll-like receptor 5 (TLR5) stimulating activity.

30. The method of claim 28, wherein the injury by exposure to radiation is gastrointestinal syndrome or hematopoietic syndrome.

31. The method of claim 28, wherein the aging is at least one selected from the group consisting of hair loss, cataracts, hernias, colitis, osteoporosis, and osteomalacia due to aging.
